(19) United States
(12) Patent Application Publication
(10) Pub. No.: US 2011/0105995 A1
(43) Pub. Date: May 5, 2011

(54) UNIFORM-SIZED, MULTI-DRUG CARRYING, AND PHOTOSENSITIVE LIPOSOMES FOR ADVANCED DRUG DELIVERY

(76) Inventors: Ting F. Zhu, Cambridge, MA (US); Jack W. Szostak, Boston, MA (US)

(21) Appl. No.: 12/863,106

(22) PCT Filed: Jan. 13, 2009
(86) PCT No.: PCT/US09/00202

§ 371 Date: Jan. 7, 2011

Related U.S. Application Data
(60) Provisional application No. 61/011,276, filed on Jan. 16, 2008.

(51) Int. Cl. A61M 37/00; A61K 9/127

(52) U.S. Cl. 604/20; 424/450

(57) ABSTRACT

Uniform-sized liposome populations can improve both the efficacy and safety of drug delivery. The present invention utilizes the techniques of extrusion with polycarbonate-membrane-based large-pore dialysis to create uniform-sized liposome populations. These uniform-sized liposome populations may comprise different sizes such that smaller liposome populations contain specific drugs that are compartmentalized within a larger liposome population. These uniform-sized liposomes can be lysed upon photoillumination and release the encapsulated drugs and/or smaller liposomes, and can be used as a new version of photodynamic therapy.

### FIELD OF INVENTION

This invention is related to the field of drug delivery systems. The delivery of drugs using liposomes have been tremendously improved by providing a system that provides the capability for multi-drug delivery, differential tissue targeting, as well as temporally sequenced drug release. For example, a topologically complex liposome is provided wherein different drugs may be separately stored within respective liposomes having either different composition and/or size. Such drugs may be preferentially released in a specific order as result of vesicular compositional and/or size differences.

### BACKGROUND

Liposomes have been widely studied drug delivery vehicles. For example, a liposome-based drug delivery systems are widely used for intravenous anticancer chemotherapy for administering drugs such as Doxil and Myocet. Abraham et al., “The liposomal formulation of doxorubicin” Methods Enzymol 391:71-97 (2005). Liposomes can also be used to deliver inhaled aerosol drugs, for example: i) insulin (Huang et al., “Pulmonary delivery of insulin by liposomal carriers” J Control Release 113:9-14 ((2006); ii) antibiotics, particularly targeting tuberculosis infection in alveolar macrophages (Vyas et al., “Aerosolized liposome-based delivery of amphotericin B to alveolar macrophages” Int J Pharm 296:12-25 ((2005); and iii) anticancer chemotherapy drugs. Verschraegen et al., “Clinical evaluation of the delivery and safety of aerosolized liposomal 9-nitro-20(s)-camptothecin in patients with advanced pulmonary malignancies” Clin Cancer Res 10:2319-2326 (2004); and U.S. Pat. No. 5,049,388 (herein incorporated by reference).

Other forms of conventional liposome drug delivery currently under development, e.g., transdermal and per oral delivery. Ulrich, A. S., “Biophysical aspects of using liposomes as delivery vehicles” Bioscience Reports 22:129-150 (2002). However, liposomes made by conventional methods are highly heterogeneous, thereby making it difficult to control the drug dose, liposome deposition, and release profile. Knight et al., “Anticancer effect of 9-nitrocamptothecin liposome aerosol on human cancer xenografts in nude mice” Cancer Chemother Pharmacol 44:177-186 (1999); and Korgel et al., “Vesicle size distributions measured by flow field-flow fractionation coupled with multiangle light scattering” Biophysical Journal 74:3264-3272 (1998).

What is needed in the art are uniform-sized liposome populations to improve drug delivery efficacy and safety for clinical use. Especially needed is the temporally controlled delivery of multiple drugs from one primary uniform-sized liposome population, wherein each primary liposome comprises a variety of differentially sized secondary liposome populations. Further, such uniform-sized liposome populations would improve the reproducibility of research results during in vitro experiments, animal tests, and clinical trials.

### SUMMARY

This invention is related to the field of drug delivery systems. The delivery of drugs using liposomes have been tremendously improved by providing a system that provides the capability for multi-drug delivery, differential tissue targeting, as well as temporally sequenced drug release. For example, a topologically complex liposome is provided wherein different drugs may be separately stored within respective liposomes having either different composition and/or size. Such drugs may be preferentially released in a specific order as result of vesicular compositional and/or size differences.

In one embodiment, the present invention contemplates a topologically complex liposome comprising a primary liposome encapsulating a first drug and a secondary liposome population, wherein said secondary liposome population encapsulates a second drug. In one embodiment, the primary liposome further encapsulates a photosensitizer. In one embodiment, the secondary liposome comprises a bilayer membrane, wherein said first drug is segregated from said second drug by said membrane. In one embodiment, the primary liposome comprises a bilayer membrane, wherein a targeting moiety is attached to said membrane. In one embodiment, the secondary liposome bilayer membrane and said primary liposome bilayer membrane comprise different lipid compositions. In one embodiment, the primary liposome bilayer membrane comprises at least one anti-fusogenic lipid, thereby improving circulation half-life as compared to conventional liposomes. In one embodiment, the anti-fusogenic lipid comprises phosphocholine. In one embodiment, the secondary liposome bilayer membrane comprises at least one fusogenic lipid. In one embodiment, the fusogenic lipid comprises phosphoethanolamine. In one embodiment, the secondary liposome encapsulates an intracellularly delivered drug. In one embodiment, the intracellularly delivered drug comprises an RNAi.

In one embodiment, the present invention contemplates a method, comprising: a) providing; i) a subject comprising a diseased tissue, wherein said tissue comprises a plurality of cells; ii) a composition comprising a topologically complex liposome comprising a primary liposome encapsulating a first drug and a secondary liposome population, wherein said secondary liposome population encapsulates a second drug; and, b) administering said composition to said subject, under conditions such that said first drug and said secondary liposome population are retained within said diseased tissue. In one embodiment, the method further comprises illuminating said delivered composition with a light source, thereby initiating a synchronized lysis of said primary liposomes. In one embodiment, the primary liposomes comprise fatty acids. In one embodiment, the primary liposomes comprise phospholipids. In one embodiment, the secondary liposome is delivered within said diseased tissue cell by an uptake mechanism. In one embodiment, the primary liposome further comprises a targeting moiety. In one embodiment, the targeting moiety comprises an antibody.

In one embodiment, the present invention contemplates a method, comprising: a) providing; i) a multilamellar lipid liposome comprising a first lipid membrane material and a first drug; ii) a second lipid membrane material; and iii) a second drug; b) extruding said multilamellar liposome to create a secondary liposome population comprising said first lipid membrane material and having a maximum average diameter; c) dialyzing said secondary liposome population, wherein said secondary liposome population further comprises a minimum average diameter; and d) encapsulating said secondary liposome population with said second lipid membrane material composition and said second drug to form a topologically complex liposome composition comprising a primary liposome population comprising said second lipid membrane material thereby encapsulating said secondary liposome population and said second drug. In one embodiment, the method further comprises dialyzing said topologically complex liposome composition, wherein unencapsulated secondary liposomes are removed from said composition. In one embodiment, the first lipid membrane material and said second lipid membrane material are identical. In one embodiment, the first lipid membrane material and said second lipid membrane material are different. In one embodiment, the lipid membrane material comprises a fatty acid. In one embodiment, the primary liposome population is of uniform size. In one embodiment, the secondary liposome population is of uniform size.

In one embodiment, the present invention contemplates a method, comprising: a) providing; i) a first lipsome population; ii) a first polycarbonate membrane comprising pores larger than 0.8 μm capable of extruding the said liposome population; and iii) a second polycarbonate membrane with pores larger than 0.8 μm capable of dialyzing liposomes smaller than the pores; b) extruding the first liposome population with the first membrane thereby creating a second liposome population; and c) dialyzing the second liposome population with the second membrane, thereby creating a uniform-sized liposome population. In one embodiment, the uniform-sized liposome population comprises a pseudo-monodisperse liposome population. In one embodiment, the uniform-sized liposomes contain a water soluble drug. In one embodiment, the uniform-sized liposomes contain human hemoglobin. In one embodiment, the uniform-sized liposomes contain recombinant human hemoglobin. In one embodiment, the uniform-sized liposomes contain animal hemoglobin. In one embodiment, the uniform-sized liposomes contain recombinant animal hemoglobin.

In one embodiment, the present invention contemplates a large uniform-sized primary liposome population. In one embodiment, the large uniform-sized primary liposome population comprises an average diameter of 0.8 μm. In one embodiment, the primary liposome population encapsulates a blood substitute. In one embodiment, the blood substitute comprises hemoglobin. In one embodiment, the primary liposome population comprises at least one phospholipid that an increases shelf-life as compared to conventional liposome populations. In one embodiment, the primary liposome population comprises at least one phospholipid that increases circulatory half-life as compared to conventional liposome populations. In one embodiment, the liposome population comprises pseudo-monodisperse liposomes.

In one embodiment, the present invention contemplates a method, comprising: a) providing; i) a topologically complex liposome composition comprising a primary liposome population encapsulating a secondary liposome population and a drug; ii) a light source, wherein said light source is capable of inducing a synchronized lysis of said primary liposome population; b) illuminating said primary liposome population with said light source, thereby inducing a synchronized lysis of said primary liposome population. In one embodiment, the lysis is mediated by an increase of osmotic pressure within said primary liposome. In one embodiment, the osmotic pressure increase is induced by a pH drop within said primary liposome. In one embodiment, the lysis is mediated by pH-sensitive phospholipids. In one embodiment, the synchronized lysis of said primary liposome population is complete within 0.4 seconds. In one embodiment, the synchronized lysis of said primary liposome population releases said drug and said second liposome population. In one embodiment, the internal osmotic pressure increase is mediated by a pH drop within said primary liposome population. In one embodiment, the pH drop is mediated by the oxidation of bicine from photooxidation within said primary liposome population. In one embodiment, the lysis is caused by pH-sensitive primary phospholipid liposomes responding to light-triggered internal pH drop.

In one embodiment, the present invention contemplates a composition, comprising an organic solvent-free primary liposome population encapsulating a first pharmaceutical agent and further encapsulating a first uniform-sized secondary organic solvent-free liposome population, wherein said first secondary liposome comprises a hollow core surrounded by a lipid bilayer such that said bilayer is not integrated with said primary liposome. In one embodiment, the first secondary liposome encapsulates a second pharmaceutical agent such that said second pharmaceutical agent is segregated from said first pharmaceutical agent. In one embodiment, the second uniform-sized secondary liposome encapsulates a third pharmaceutical agent such that said third pharmaceutical agent is segregated from said first pharmaceutical agent and said second pharmaceutical agent, wherein said second secondary liposome comprises a hollow core surrounded by a lipid bilayer such that said bilayer is not integrated with said first secondary liposome. In one embodiment, the primary liposome further comprises a photosensitizer. In one embodiment, the photosensitizer comprises bicine. In one embodiment, the second secondary liposome and said first secondary liposome comprise different lipid compositions. In one embodiment, the primary liposome comprises a first average diameter having a standard deviation of between approximately 15-30%. In one embodiment, the first secondary liposome comprises a second average diameter, wherein said second average diameter is less than said first average diameter. In one embodiment, the second secondary liposome comprises a third average diameter, wherein said third average diameter is less than said second average diameter.

In one embodiment, the present invention contemplates a method, comprising: a) providing; i) a subject comprising a diseased tissue; ii) a composition comprising an organic solvent-free primary liposome population comprising a first pharmaceutical agent and a photosensitizer, wherein said primary liposome encapsulates a first uniform-sized organic solvent-free secondary liposome population comprising a second pharmaceutical agent, and further encapsulating a second uniform-sized solvent-free secondary liposome comprising a third pharmaceutical agent; and, b) administering said composition to said subject, under conditions such that said primary liposome is retained within said diseased tissue; and c) illuminating said diseased tissue with a radiation source, thereby initiating a synchronized lysis of said primary liposomes. In one embodiment, the first pharmaceutical agent is released before said second pharmaceutical agent and said third pharmaceutical agent. In one embodiment, the second pharmaceutical agent is released before said third pharmaceutical agent. In one embodiment, the radiation source is visible light at approximately 250 mW intensity. In one embodiment, the radiation source comprises an X-ray source. In one embodiment, the radiation source comprises a gamma ray source.

In one embodiment, the present invention contemplates a method, comprising: a) providing; i) a multilamellar liposome comprising a first lipid membrane material composition and a first pharmaceutical agent; ii) a second lipid membrane material composition; and iii) a second pharmaceutical agent; b) extruding said multilamellar liposome to create a uniform-sized organic solvent-free first secondary liposome population having a maximum average diameter; c) dialyzing said first secondary liposome, wherein said first secondary liposome further comprises a minimum average diameter; and d) encapsulating said first secondary liposome with said second lipid membrane material composition and a second pharmaceutical agent to form an organic solvent-free primary liposome population comprising said first secondary liposome, wherein said first secondary liposome is not integrated with said primary liposome. In one embodiment, the method further comprises extruding said primary liposome to create a uniform-sized primary liposome having a maximum average diameter. In one embodiment, the method further comprises dialyzing said uniform-sized primary liposome, wherein said uniform-sized primary liposome further comprises a minimum average diameter. In one embodiment, the multilamellar liposome further comprises a photosensitizer. In one embodiment, the lipid membrane material comprises a fatty acid.

### DEFINITIONS

The term “topologically complex liposome” as used herein, refers to any composition comprising a liposome-within-a-liposome such that a larger liposome (i.e., for example, a primary liposome) encapsulates a smaller liposome (i.e., for example, a secondary liposome). The primary and secondary liposomes have separate and independent lipid bilayer membranes that do not become attached or integrated; occasional incidental contact may occur as a result of random Brownian motion. These independent lipid bilayer membranes allow the secondary liposome to encapsulate different components than the primary liposome without cross-contamination.

The term “primary liposome” as used herein, refers to any liposome that encapsulates at least one smaller liposome (i.e., for example, a secondary liposome), wherein the lipid bilayer membrane of the primary liposome is not attached to, or integrated with, the lipid bilayer membrane of the encapsulated smaller liposome.

The term “secondary liposome” as used herein, refers to any liposome that is encapsulated by a larger liposome (i.e., for example, a primary liposome), wherein the lipid bilayer membrane of the secondary liposome is not attached to, or integrated with, the lipid bilayer membrane of the encapsulating larger liposome.

The term “population” as used herein, refers to a plurality of individual liposomes contained within the same solution. Such liposomes may include, but are not limited to, topologically complex liposomes, primary liposomes, and/or secondary liposomes.

The term “drug” or “pharmaceutical agent” as used herein, refers to any compound, molecule, peptide, protein, hormone etc. that may be administered to a subject that is capable of having a medicinal benefit. Such drugs may be hydrophobic or hydrophilic and capable of becoming encapsulated within a topologically complex liposome, a primary liposome, and/or a secondary liposome. Such drugs may be carried within these liposomes either within the encapsulated interior space or integrated within the lipid bilayer membrane.

The term “encapsulates” or “encapsulating” as used herein, refers to any lipid bilayer membrane the completely surrounds (i.e., for example, by forming a spherical shape) at least one drug and/or liposome.

The term “photosensitizer” as used herein, refers to any compound that facilitates and/or potentates the efficacy of an administered drug following exposure to a light and/or radiation source. For example, a photosensitizer may produce radical oxygen species that result in sodium ion influx into a liposome, thereby resulting in liposomal lysis to release liposomal contents (i.e., for example, other liposomes and/or drugs). Alternatively, a photosensitizer may have intrinsic therapeutic benefits.

The term “bilayer membrane” as used herein, refers to any tail-tail arrangement of fatty acids thereby forming a stable fatty acid aggregation that spontaneously create liposomes.

The term “segregated” as used herein, refers to any physical separation of at least two different drugs and/or liposomes of different compositions and/or sizes. The physical separation is such that contact between these different drugs and/or liposomes are prevented. For example, the independent bilayer membrane of an encapsulated secondary liposome provides a physical separation from an encapsulating primary liposome.

The term “targeting moiety” as used herein, refers to any compound capable of attaching to the outer membrane layer of a liposome, wherein the compound has an affinity for a specific tissue or cell. For example, a targeting moiety may comprise an antibody directed towards a cell surface receptor. Alternatively, a targeting moiety may comprise a compound or molecule capable of recognizing a specific cell internalization receptor.

The term “attached” or “attaching” as used herein, refers to any interaction between a two compositions and/or compounds such that a stable complex is formed. Such a complex may be stabilized by non-covalent interactions, covalent interactions, electrostatic forces, Van Der Waals forces, hydrophobic interactions etc.

The term “lipid composition” as used herein, refers to the components of a bilayer membrane. Such lipids may include, but are not limited to, fatty acids, triglycerides, diglycerides, monoglycerides, cholesterol, lipoproteins, and/or glycolipoproteins.

The term “fatty acid” as used herein, refers to a carbon chain molecule terminating a carboxylic acid having between one and twenty-five carbons. Such carbon chain molecules may be unsaturated (i.e., for example, containing at least one double bond) or unsaturated (i.e., for example, not containing any double bonds).

The term “pseudo-monodispersity” as used herein, refers to uniform-sized liposomes containing pharmaceutical agents, mixed with liposomes (i.e., for example, of the same of different sizes, mono- or poly-disperse) that do not contain any pharmaceutical agents and/or perform any biological function.

The term “subject” or “patient” as used herein, refers to any living organism to which a topologically complex liposome composition may be administered. For example, a living organism may include mammals and/or non-mammals. Mammals may include, but are not limited to, humans, dogs, cats, cattle, sheep, pigs, and/or goats. Non-mammals may include, but are not limited to, reptiles, birds, and/or fish. Alternatively, a living organism may include microbial species including, but not limited to, bacteria, viruses, fungi, and/or molds.

The term “diseased tissue” as used herein, refers to any tissue that may be infected, injured, expressing a genetic abnormality, and/or subject to dysregulation caused by an altered level of hormone or other bodily regulatory compound. A diseased tissue would be expected to have reduced functionality. Such a diseased tissues may be confirmed by biopsy samples, blood samples, and/or imaging techniques (i.e., for example, MRI, CAT scan).

The term “normal tissue” or “non-diseased tissue” as used herein, refers to any tissue having a functionality that is within medically accepted ranges.

The term “cells” as used herein, refers to any small, usually microscopic, mass of protoplasm bounded externally by a semipermeable membrane (i.e., for example, a lipid bilayer), usually including one or more nuclei and various nonliving products, capable alone or interacting with other cells of performing all the fundamental functions of life, and forming the smallest structural unit of living matter capable of functioning independently. A tissue comprises a plurality of specialized cells.

The term “administering” as used herein, refers to any method by which a topologically complex liposome composition may be provided to a patient and/or subject. Such administering may be parenteral or non-parenteral. Non-parenteral administration includes, but is not limited to, oral, intragastric intubation, or intranasal intubation. Parenteral administration includes, but is not limited to, intravenous injection, intramuscular injection, intraperitoneal injection, topical (i.e., for example, by gel, cream, and/or lotion), intranasal (i.e., for example, by spray), intrapulmonary (i.e., for example, by aerosol), or suppository.

The term “retained” as used herein, refers to the capture, uptake, or otherwise holding a topologically complex liposome at, in, or near a tissue or cell.

The term “illuminating” as used herein, refers to any exposure of a tissue, cell, and or liposome to a light source and/or a radiation source.

The term “light source” or “radiation source” as used herein, refers to any device and/or compound capable of producing electromagnetic energy of specific wavelengths. For example, the wavelengths may be in the infrared light spectrum, the visible light spectrum, the ultraviolet light spectrum, and/or the X-ray spectrum.

The term “visible light” as used herein, refers to any electromagnetic energy having a wavelength of approximately 400 nm for violet light to about 700 nm for red light.

The term “X-ray source” as used herein, refers to any electromagnetic energy having a wavelength of less than 100 angstroms.

The term “gamma ray source” as used herein, refers to any photon emitted by a radioactive source (i.e., for example cobalt 60; ⁶⁰Co).

The term “synchronized lysis” as used herein, refers to the lysis of any liposomal population in response to a single event, wherein substantially all liposomes (i.e., for example, 98%-100% of liposomes in the event area) are lysed within 0.4 seconds of the initiation of the event (i.e., for example, illumination). Such liposomal population may contain membranes comprising for example, either fatty acids or phospholipids.

The term “exploding lipsome” as used herein, refers to any liposome that is capable of lysing as a result of a generalized and complete membrane breakdown. Although it is not necessary to understand the mechanism of an invention, it is believed that such a breakdown can include, but not be limited to: 1) photoillumination→p internal pH drop→p Na⁺ influx to balance pH→internal osmotic increase->non-pH sensitive liposome lysis; or 2) photoillumination→internal pH drop→pH-sensitive primary phospholipid liposome lysis.

The term “pH sensitive” as used herein refers to a molecule which changes in conformation or other properties in response to changes in pH of the surrounding environment. As used herein, the term further refers to a molecule whose conformation or properties changes as pH decreases from 7.4 to 3.5-6.5.

The term “released” or “releasing” as used herein, refers to the delivering of encapsulated drugs and/or secondary liposomes in, at, or near a bodily tissue or cell resulting from the lysis of a primary liposome.

The term “delivered” or “delivering” as used herein, refers to any placement of a drug or secondary liposome within the immediate vicinity (i.e., for example, at, in or near) of a bodily tissue or cell. For example, a drug may be delivered to a tissue following the lysis and subsequent release of the drug from a liposome.

The term “uptake mechanism” as used herein, refers to any process by which a drug and/or a liposome is translocated across a cell bilayer membrane and into the intracellular space (i.e., for example, the cytosol). For example, such uptake mechanisms may include, but are not limited to, phagocytosis, active membrane transport proteins, and/or internalization receptors.

The term “antibody” as used herein, refers to any protein of high molecular weight that are produced normally by specialized B cells after stimulation by an antigen and act specifically against the antigen in an immune response. Typically, antibodies comprise four subunits including two heavy chains and two light chains. Alternatively, fragments of the high affinity regions may be used.

The term aptamer” as used herein, refers to oligonucleic acid or peptide molecules that bind a specific target molecule.

The term “multilamellar lipid liposome” as used herein, refers to a lipid liposome comprising a plurality of bilayer membranes within the interior space, a portion of which are integrated with the liposome bilayer membrane layer. Such multilamellar lipid liposomes do not segregate a liposome into different liposomes, and are created using methods that result in a uniform distribution of encapsulated compounds irrespective of the bilayer membranes within the interior space.

The term “extruding” or “extrusion” as used herein, refers to any process and/or device that forces, presses, or pushes out a liposomal population through a membrane and/or sieve to create a liposomal population of a maximum average diameter.

The term “dialyzing” or “dialysis” as used herein, refers to any method and/or device that separates substances in solution by means of their unequal diffusion through semipermeable membranes and/or membranes having a specific pore size. For example, a liposomal population may be dialyzed to create a liposomal population having a minimum average diameter. Further, dialysis of a newly formed topologically complex liposome composition would remove encapsulated components (i.e., for example, drugs and/or secondary liposomes) from the composition.

The term “average diameter” as used herein, refers to a statistical determination of a liposomal population measured by the distance across the longest portion of the liposome (i.e., for example, the equator). For example, an average may be determined by summing a plurality of individual values and dividing by the number of values.

The term “unencapsulated” as used herein, refers to any component (i.e., for example, a drug and/or secondary liposome) that remains outside a liposome immediately following the creation of a liposome population.

The term “uniform size” as used herein, refers to a specific liposome population having pre-determined average diameter range (i.e., for example, having a specific maximum average diameter and a specific minimum average diameter). Such uniform-sized liposome populations can be created by the combination of extrusion with dialysis.

The term “complete” as used herein, refers to the cessation of liposomal lysis in response to a single event (i.e., for example, illumination). For example, such completion may occur after substantially all liposomes (i.e., for example, 98%-100%) have lysed in response to the single event.

The term “organic solvent-free” as used herein, refers to any liposome population that was created without the use of organic solvents. It is not intended that an organic solvent-free liposome population be created with an organic solvent, wherein the organic solvent is evaporated. Liposome populations subjected to the evaporation of organic solvents are known to still comprise sufficient organic solvents to induce tissue toxicity upon clinical administration.

The term “encapsulated aqueous compartment” as used herein, refers to the interior space within a liposome that is created following the encapsulation of a solution, wherein the solution may, or may not, comprise drugs and/or secondary liposomes.

The term “integrated” or “integrating” as used herein, refers to at least two bilayer membranes that have formed or blended into a unified whole.

The term “intracellular delivery of drugs” as used herein, refers to a method of delivering drugs into the cell cytoplasm, lysosome, endosome, mitochondrion, and/or nucleus, to achieve certain therapeutic effects. The drugs to be delivered by such means may include, but are not limited to, RNA molecules, DNA molecules, large-molecule drugs (i.e., proteins), high-systemic-toxicity drugs, and drugs that have short lifetime in blood circulation.

The term “RNA interference (RNAi)” as used herein, refers to a mechanism that inhibits gene expression by causing the degradation of specific RNA molecules or hindering the transcription of specific genes. Currently, such small RNA molecules used for RNAi therapy may include, but are not limited to, siRNA and microRNA.

The term “fusogenic lipid” as used herein, refers to lipid that enhances cellular uptake through liposome-cell fusion or endocytosis. Such fusogenic lipid may include, but are not limited to, DOPE. Torchilin, V. P., “Recent approaches to intracellular delivery of drugs and DNA and organelle targeting” Annu Rev Biomed Eng 8: 343-75 (2006); and de Fougerolles et al., “Interfering with disease: a progress report on siRNA-based therapeutics” Nat Rev Drug Discov 6:443-453 (2007).

The term “fusogenic liposome” as used herein, refers to liposomes that have the ability to be fused or endocytosed into target cells, to deliver drugs intracellularly. Such a process can be achieved through, but are not limited to, cell endocytosis by ligand-receptor recognition, electrostatic/hydrophobic interaction between the liposome and cell membranes, viral-antigen-facilitated liposome-cell fusion and/or endocytosis, the use of fusogenic lipid in the liposome composition, and antibody/aptamer-mediated liposome attachment to cells.

The term “long-circulating liposome” as used herein, refers to liposomes that have long life time in the blood circulation, i.e., for example, by avoiding immune recognition and endocytosis. Such long-circulating effect may be achieved through, but are not limited to, liposome surface modification (PEGylation) and/or liposome sizing.

### BRIEF DESCRIPTION OF THE DRAWINGS

FIG. 1 presents one embodiment of a large-pore dialysis setup.

FIG. 1A: Original membranes on a dialysis cassette were replaced by wetted polycarbonate track-etched membranes.

FIG. 1B: A 300-400 μl extruded sample was loaded to the center of the dialysis cassette.

FIG. 1C: The dialysis cassette sealed using clamps.

FIG. 1D: Approximately 30 ml of washing buffer was used in each round of dialysis, which just submerged the laid-down dialysis cassette in a 150 ml cup.

FIG. 2 presents one embodiment of a method of making a 3˜5 μm uniform-sized oleic acid liposome population.

FIG. 2A: A polydisperse oleic acid liposome population after being extruded with a 5 μm-pore-size membrane.

FIG. 2B: The corresponding size distribution of the population in FIG. 2A.

FIG. 2C: A uniform-sized oleic acid liposome population having a narrow range of approximately 3-5 μm, after the polydisperse liposome population in FIG. 2A underwent 12 rounds of large-pore dialysis with 3-μm-pore-size membranes.

FIG. 2D: The corresponding size distribution of the oleic acid liposome population in FIG. 2C.

FIG. 3 presents one embodiment of a method for making a 0.8-1 μm uniform-sized POPC liposome population.

FIG. 3A: A polydisperse POPC liposome population after being extruded with a 1 μm pore-sized membrane.

FIG. 3B: The corresponding size distribution of the polydisperse liposome population in FIG. 3A.

FIG. 3C: A uniformed sized POPC liposome population having a narrow range of approximately 0.8-1 μm, after the polydisperse POPC liposome population in FIG. 3A underwent 12 rounds of dialysis with 0.8-μm-pore-size membranes.

FIG. 3D: The corresponding size distribution of the POPC liposome population in FIG. 3C.

FIG. 4 illustrates one embodiment of a method for encapsulating a uniform-sized rhodamine-DHPE labeled liposomes. Multilamellar oleic acid liposomes containing a rhodamine-DHPE fluorescent label were extruded creating a secondary liposome population having a 100 nm average diameter. This secondary liposome population was re-encapsulated using a solution of oleic acid followed by large-pore dialysis (i.e., 3 μm) to eliminate the unencapsulated rhodamine-DHPE labeled liposomes of 3 μm or less, thereby creating a uniform-sized primary liposome population having an average diameter of between larger than 3 μm.

FIG. 5 illustrates one embodiment of a method for making a multi-drug delivery topologically complex liposome population. Multilamellar oleic acid liposomes containing drug B (green) are extruded creating a secondary liposome population having a 100 nm average diameter. Unencapsulated drug B is removed from the liposomal population by size-exclusion chromatography and/or dialysis. A solution comprising drug A (blue) and additional oleic acid re-encapsulates the secondary liposome population to create a primary liposome population. Large-pore dialysis (3 μm) is then performed to eliminate the unencapsulated drug B-containing liposomes and free drug A in the solution to create a uniform-sized topologically complex primary liposome population have an average diameter of between larger than 3 μm.

FIG. 6 shows an exemplary photomicrograph showing 100 nm diameter rhodamine-DHPE labeled oleic acid secondary liposomes (bright spots) within primary liposomes that are greater than 3 μm in diameter. The outer layer membrane of the primary liposome is not labeled by any fluorescent dye and, therefore, invisible by fluorescent microscopy.

FIG. 7 presents one embodiment for a pulmonary administration of a topologically complex primary liposome for drug delivery.

FIG. 8 shows exemplary data of synchronized release from a topologically complex primary liposome population. Note that the encapsulated fluorescent dye as well as the encapsulated secondary liposomes were released.

FIG. 8A: Primary liposome fluorescence at the moment of illumination.

FIG. 8B: Primary liposome fluorescence 0.4 sec after the illumination.

FIG. 9 presents exemplary data showing a photoillumination-induced radical-oxygenation product of bicine identified by mass spectroscopy.

FIG. 9A: A unilluminated mixture of 0.2 M pH 8.5 bicine buffer, 2 mM HPTS, and 80 mM H2O2 showing a single peak, corresponding to the ionized form of bicine.

FIG. 9B: The same mixture as in FIG. 9A after illumination with a 480±20 nm light source for 30 min showing an additional peak corresponding to the radical-oxygenation product of bicine. Its predicted chemical structure is also shown.

FIG. 10 illustrates size-dependency of the photoactivated liposome lysis, which was verified by experimental data (not shown).

FIG. 11 provides an illustration comparing the mechanisms of action of conventional photodynamic therapy and topologically complex primary liposome photodynamic therapy. The conventional photodynamic therapy relies on massive reactive oxygen species release to induce direct tissue-damaging effects. The topologically complex primary liposome photodynamic therapy relies on minimal reactive oxygen species generation (within the liposome) to initiate a series of chemical/physical processes leading to liposome lysis and drug release.

FIG. 12 provides an exemplary schematic for “radiation-dynamic” therapy using topologically complex primary liposome populations. For example, the liposomes are administered into blood circulation and a focalized radiation source (i.e., for example, X-ray and/or gamma-ray) induces synchronized liposome lysis providing the localized release of a therapeutically effective drug and/or secondary liposomes comprising a different therapeutically effective drug.

FIG. 13 presents various prior art liposome compositions.

FIG. 13A shows various solvent spherules evaporated together to form a liposome.

FIG. 13B shows a liposome encapsulating oil-based particle nanosuspensions.

FIG. 13C shows a liposome encapsulating a central nanocore particle.

FIG. 14 illustrates one embodiment of a topologically complex liposome comprising a primary liposome encapsulating a secondary liposome population.

FIG. 15 illustrates one embodiment of a topologically complex liposome comprising a primary liposome encapsulating fusogenic secondary liposomes, wherein the secondary liposome may encapsulate an RNAi.

FIG. 16 presents exemplary data showing synchronized lysis of phospholipid vesicles. Panel A: A representative phospholipid vesicle before illumination. Panel B: A representative disrupted phospholipid vesicle within approximately 0.5 second after illumination.

### DETAILED DESCRIPTION

This invention is related to the field of drug delivery systems. The delivery of drugs using liposomes have been tremendously improved by providing a system that provides the capability for multi-drug delivery, differential tissue targeting, as well as temporally sequenced drug release. For example, a topologically complex liposome is provided wherein different drugs may be separately stored within respective liposomes having either different composition and/or size. Such drugs may be preferentially released in a specific order as result of vesicular compositional and/or size differences.

### I. Liposomes And Drug Delivery

Liposomes have been widely studied drug delivery vehicles. For intravenous liposomal drug delivery, the liposome size distribution was noted to affect drug efficacy. Nagayasu et al., “The size of liposomes: a factor which affects their targeting efficiency to tumors and therapeutic activity of liposomal antitumor drugs” Adv Drug Deliv Rev 40:75-87 (1999). It is suspected that the selective accumulation of liposomes in tumors might be size-dependent, because the tumor capillaries have relatively larger pore sizes (i.e., for example, 100˜700 nm, depending on the type of tumor) as compared to normal blood vessels, which are typically less than 50 nm. Consequently, it has been reported that liposomes in a certain size range can penetrate the tumor capillaries more easily, for which the optimal diameter measured was approximately between 90˜200 nm. Liu et al., “Role of liposome size and RES blockade in controlling biodistribution and tumor uptake of GM1-containing liposomes” Biochim Biophys Acta 1104:95-101 (1992). It is believed that smaller liposomes will be able to penetrate the tumor capillaries but will not be easily trapped in the tumor, thereby compromising their retention rate. In addition, smaller liposomes may be able to penetrate normal tissue capillaries from blood circulation more easily, thereby causing toxicity to normal tissue. Others have shown that drug release profiles from liposomes in vivo might also be size-dependent. Nagayasu et al., “Effect of vesicle size on in vivo release of daunorubicin from hydrogenated egg phosphatidylcholine-based liposomes into blood circulation” Biol Pharm Bull 18:1020-1023 (1995). For inhaled liposomal drug delivery, the ideal liposome size is 1˜3 μm because particles in this size range can be delivered into the deep lung more effectively and avoid phagocytic clearance from the lung periphery. Dhand, R., “New frontiers in aerosol delivery during mechanical ventilation” Respir Care 49:666-677 (2004); Edwards et al., “Recent advances in pulmonary drug delivery using large, porous inhaled particles” J Appl Physiol 85:379-385 (1998); and Verschraegen et al., “Clinical evaluation of the delivery and safety of aerosolized liposomal 9-nitro-20(s)-camptothecin in patients with advanced pulmonary malignancies” Clin Cancer Res 10:2319-2326 (2004).

### I. Liposomal Multi-Drug Delivery Systems

Clinical use of liposomes can be optimized by using uniform-sized liposome populations. For example, in liposomal intravenous drug delivery, size has been shown as a factor in determining drug delivery efficacy. Nagayasu et al., “The size of liposomes: a factor which affects their targeting efficiency to tumors and therapeutic activity of liposomal antitumor drugs” Adv Drug Deliv Rev 40:75-87 (1999). For example, size may affect the stability of liposomes in blood circulation, wherein liposomes made of polyethylene glycol-binding-phospholipids with a diameter of 100˜200 nm were shown to have improved stability. In addition, the selective accumulation of liposomes in tumors is size-dependent, because the tumor capillaries have relatively larger pore sizes (100˜700 nm, depending on the type of tumor) compared to the normal blood vessels (typically less than 50 nm). Liposomes in a certain size range can penetrate the tumor capillaries more easily than the normal tissue capillaries, for which the optimal diameter measured is 90˜200 nm. Liu et al., “Role of liposome size and RES blockade in controlling biodistribution and tumor uptake of GM1-containing liposomes” Biochim Biophys Acta 1104:95-101 (1992). Alternatively, smaller liposomes that can penetrate the tumor capillaries may not be easily trapped within the tumor, thereby reducing liposome retention. Also, smaller liposomes can penetrate the normal tissue capillaries from blood circulation more easily, which could cause toxicity to the normal tissue.

Drug release profiles have also been shown to be dependent upon liposome average diameter. Nagayasu et al., “Effect of vesicle size on in vivo release of daunorubicin from hydrogenated egg phosphatidylcholine-based liposomes into blood circulation” Biol Pharm Bull 18:1020-1023 (1995). For example, ideal liposome sizes for inhaled liposome drug delivery have been suggested to range between 1˜3 μm because particles in this size range have prolonged drug release time and can avoid phagocytic clearance from the lung periphery. Dhand R., “New frontiers in aerosol delivery during mechanical ventilation” Respir Care 49:666-677 (2004); and Edwards et al., “Recent advances in pulmonary drug delivery using large, porous inhaled particles” J Appl Physiol 85:379-385 (1998).

A. Multivesicular Liposomal Drug Delivery Systems

The incorporation of different agents into a liposome by separately encapsulating nanosuspensions comprising “liquid and/or solid particles has been reported. Solis et al., “Encapsulation Of Nanosuspensions In Liposomes And Microspheres” United States Patent Application Publication No. 2003/0096000; and Kim S., “Heterovesicular Liposomes” U.S. Pat. No. 5,422,120 (both herein incorporated by reference). These methods utilize an organic solvent and require multiple “water-in-oil-in-water” emulsions. Each separate emulsion contains a drug that is subsequently encapsulated into a single liposome, followed by evaporation of the organic solvent. The evaporation step creates a ‘solvent spherule’ (i.e., an amorphous mass of assembled lipid molecules) wherein the various liposomes fuse together thereby forming a network of interconnected chambers containing a mixture of drug nanosuspensions. The compositions do not comprise a primary liposome encapsulating a secondary liposome wherein the secondary liposome comprises a hollow core surrounded by a membrane bilayer such that said bilayer is not integrated with said primary liposome such that a first drug (within the primary liposome) may be segregated from a second drug (within a secondary liposome).

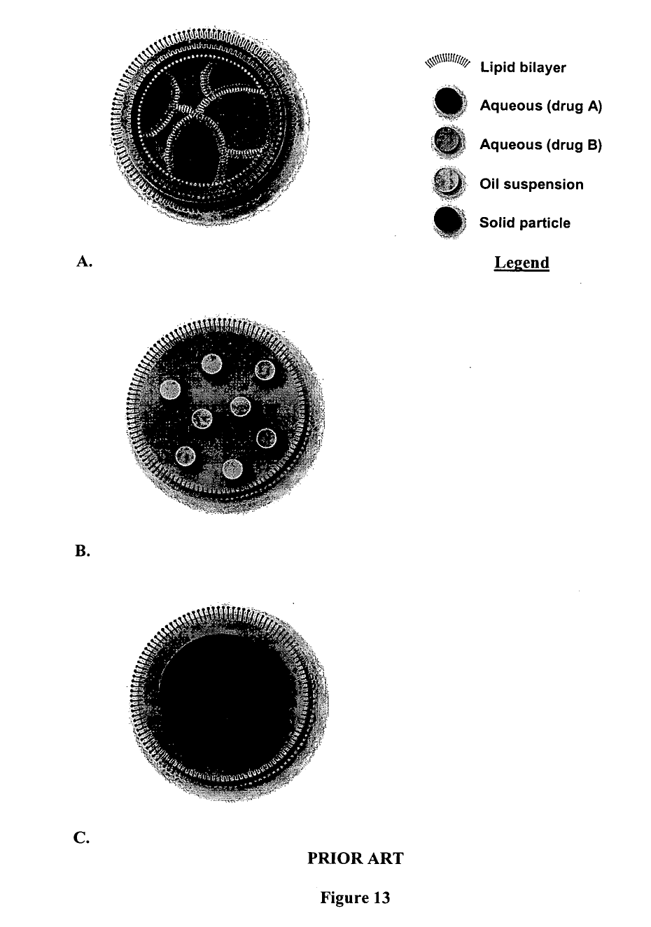

The compositions described in the '120 patent, contain two drugs which are simply blended and encapsulated together within the lipid-solvent droplet. See, FIG. 13A When the two drugs are released, they are released simultaneously; and they cannot be “programmed” to release one after another (i.e., sequentially). Many clinical applications, especially for treating cancer, are treated most effectively following a programmable sequential release of multiple drugs. This liposomal multi-drug delivery system cannot achieve the sequential drug release because it simply administers two different drugs at the same time. These liposomes are produced by making aqueous emulsions in an organic solvent and then evaporating the organic solvent. However, by using the described techniques, residual organic solvent contamination is known to exist. However, recent studies show that liposomes made by similar methods are contaminated by the organic solvent. Utada et al., “Monodisperse double emulsions generated from a microcapillary device” Science 308:537-541 (2005). The remaining organic solvent layer within the liposome lipid membrane, is often hazardous to biological tissue. See, FIG. 13A. This organic layer also makes the liposome hydrophobic, thereby reducing the ability for tissues and/or cell to absorb the liposome for localized drug delivery. Because of the safety related issues, liposomes made by these methods are not clinically used for drug delivery, despite the over two decades' research on similar techniques.

The compositions within the '000 application are not multi-drug delivery liposomes. These liposomes are designed to deliver single hydrophobic drugs using hydrophobic oil suspensions (nanosuspensions) encapsulated within liposomes. See, FIG. 13B. Consequently, these compositions can only deliver hydrophobic drugs (a significant minority of all known drugs) carried within an oil suspension. It should be noted that most clinically used drugs are water-soluble and thus cannot be delivered using this liposome composition. Further, this design requires the use of an organic solvent for making the oil suspensions and suffer the same disadvantages as described above for the '120 patent. In addition, the drug release profile from these nanosuspensions is also very slow (˜30 days), which is undesirable in many clinical applications. Because of these safety concerns and limited therapeutic effectiveness, drug delivery systems involving oil suspensions have not been clinically approved or used.

Liposomes having oil suspensions within a lipid bilayer membrane tend to be very unstable, because the oil suspensions fuse with each other or into the lipid bilayer. Practically, this will greatly limit the shelf life of the product. In contrast, some embodiments of the present invention contemplate improved liposome population stability provided by the negatively-charged lipid bilayer membranes, thereby creating a stable liposome population for at least several months.

Consequently, previous publications regarding a liposome having one or more drug agents are subject to many disadvantages including, but not limited to: i) using oil suspensions to deliver drugs is often hazardous, ineffective, and not clinically approved or used; ii) only a minority of drugs are hydrophobic that can be dissolved and delivered by oil suspensions; and iii) if multiple drugs were to be delivered using this design, they would not physically separated by lipid bilayer membranes, and therefore can dissolve and/or react with each other. Further, these previous publications provide no guidance to utilize the unique procedures of making topologically complex liposomes as contemplated herein.

Drug delivery compositions capable of sequential delivery of two different therapeutic agents have been reported that require a “nanocore” bound with a first agent inside a lipid liposome containing a second therapeutic agent. See, FIG. 13C. The nanocores are made by associating drugs with a polymer matrix (i.e., by covalent or non-covalent bonding), thereby allowing a slow and controlled release. The nanocores can be surrounded by multiple types of pharmaceutically-acceptable lipid liposomes, including liposomes. Sengupta et al. “Nanocell Drug Delivery System”, United States Patent Application Publication Number 2007/0053845. The compositions do not comprise a primary liposome encapsulating a secondary liposome wherein the secondary liposome comprises an encapsulated aqueous compartment surrounded by a membrane bilayer such that said bilayer is not integrated with said primary liposome such that a first drug (within the primary liposome) may be segregated from a second drug (with the secondary liposome).

The '845 patent publication teaches that the chemotherapeutic drug has to be covalently conjugated to the solid particle, otherwise it will be dissolved into the aqueous compartment. Clearly, this technology is limited to only those chemotherapeutic drugs that can be covalently conjugated to a biodegradable nanoparticle (i.e., drugs must be chemically modified to form a covalent bond with a specific biodegradable polymer, yet still preserve effectiveness and safety for clinical use). So far, it is believed that only one nanoparticle-conjugated drug (doxorubicin) has been used in published research, despite the various potential applications mentioned in the patent. Another disadvantage of a central-core based liposome drug delivery platform is that in order to release the covalently conjugated drug, the biodegradable nanoparticle has to be degraded in the tissue. This makes the drug release profile very slow (˜15 days), which is undesirable in many clinical applications. This also adds an uncontrollable variables to the drug release including, but not limited to, tissue fluid flow conditions wherein the biodegradable nanoparticles can be degraded slower/faster in certain tissues, and most biodegradable materials can trigger immune responses during degradation, which may cause severe complications in patients with certain diseases. Additionally, the techniques described in the '845 patent publication cannot sufficiently eliminate unencapsulated solid particles attached to chemotherapeutic drugs and are potentially harmful to normal tissues.

B. Multicompartmental Liposomal Drug Delivery Systems

Liposomes have been produced that have multiple concentric bilayer membranes surrounding a central lipophilic core where various drugs and biologically active agents may be incorporated between the various bilayers. Foldvari M., “Method For Preparing Biphasic Multilamellar Lipid Vesicles” U.S. Pat. No. 5,993,851. These compositions, however, do not segregate different drugs between the various bilayer compartments and clearly show that the same biologically active ingredients are present in both the central core and the peripheral compartments. The compositions do not comprise a primary liposome encapsulating a secondary liposome wherein the secondary liposome comprises a hollow core surrounded by a membrane bilayer such that said bilayer is not integrated with said primary liposome such that a first drug (within the primary liposome) may be segregated from a second drug (with the secondary liposome).

Alternatively, multivesicular liposomes (i.e., vesosomes) have been proposed for use as a drug delivery system. In creating vesosomes, small unilamellar vesicles (SUVs) are first created comprising at least one drug. These SUVs are aggregated by surface bound molecular recognition receptors (i.e., for example, biotin) and then admixed with chochleated cylinders under conditions such that the cylinders unroll and encapsulate the vesicles. Zasadzinski et al., “Bilayer Structure Which Encapsulates Multiple Containment Units And Uses Thereof” U.S. Pat. No. 6,221,401. This process does not result in a vesosome can contain more than one agent simultaneously.

Vesosomes have been described that can encapsulate different materials and have different bilayer compositions. These methods of creating bilayer compartments require an organic solvent (i.e., for example, ethanol) to a variety of saturated phospholipids. Kisak et al., “The vesisome—A multicompartment drug delivery vehicle” Current Medicinal Chemistry 11, 199-219 (2004). The resulting vesosomes can entrap other vesicles, biological macromolecules, or colloidal particles for use in various applications including drug delivery, but do not segregate like particles from other particles. Vesosomes do not comprise a primary liposome encapsulating a secondary liposome wherein the secondary liposome comprises a hollow core surrounded by a membrane bilayer such that said bilayer is not integrated with said primary liposome such that a first drug (within the primary liposome) may be segregated from a second drug (with the secondary liposome).

Multicompartmentalized liposomes capable of drug delivery have also been produced that have external surface recognition molecules. The method that create a multicompartmental liposomes encapsulate small liposomes using un-rolled multilamellar sheets (i.e., a technology similar to vesosomes). These multicompartmentalized liposomes do not comprise a primary liposome encapsulating a secondary liposome wherein the secondary liposome comprises a hollow core surrounded by a membrane bilayer such that said bilayer is not integrated with said primary liposome such that a first drug (within the primary liposome) may be segregated from a second drug (with the secondary liposome). Paleos et al. “Interaction between complementary liposomes: A process leading to multicompartment systems formation” Journal of Molecular Recognition 19:60-67 (2006).

Multicompartmentalized liposomes have been suggested to be useful as a single-vehicle delivery system for combinatory chemotherapeutic regiments and multimodal agents. A multi-compartmental liposome (MCL) was produced following a 24 incubation of a solution comprising small unilamellar vesicles (SUV) and large unilamellar vesicles (LUV). A photomicrographic analysis demonstrated that the MCLs and SUV/LUVs underwent partial membrane fusion. Al-Jamal et al., “Construction of nanoscale multicompartment liposomes for combinatory drug delivery” International Journal of Pharmaceutics 331:182-185 (2007). The compositions do not comprise a primary liposome encapsulating a secondary liposome wherein the secondary liposome comprises a hollow core surrounded by a membrane bilayer such that said bilayer is not integrated with said primary liposome such that a first drug (within the primary liposome) may be segregated from a second drug (with the secondary liposome).

C. Topologically Complex Liposomes

In one embodiment, the present invention contemplates compositions and methods related to topologically complex liposomes that physically separate multiple drugs. In one embodiment, a topologically complex liposome population improves existing liposomal drug delivery systems by having the capability to release multiple drugs sequentially. Some liposome drug delivery systems utilize nanoparticles as a drug carriers, but this approach limits the variety of drugs that can be delivered because they must be covalently attached to the nanoparticle. In one embodiment, the present invention contemplates a topologically complex drug-encapsulating liposome that delivers drugs which are free in solution and are not covalently attached to a carrier.

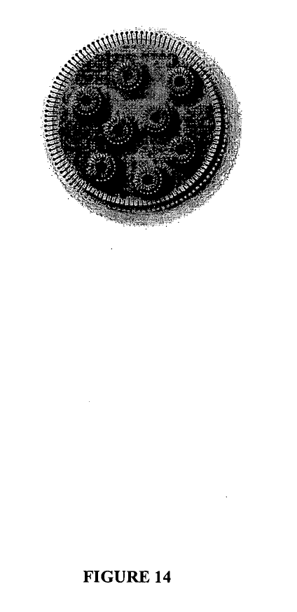

In one embodiment, the present invention contemplates a topologically complex liposome drug delivery system comprising a primary liposome population encapsulating at least one secondary liposome population. In one embodiment, the secondary liposome population comprises a first drug. In one embodiment, the primary liposome population comprises a second drug. In one embodiment, the secondary liposome population is not contaminated by the second drug. In one embodiment, the primary liposome population is not contaminated by the first drug. See, FIG. 14.

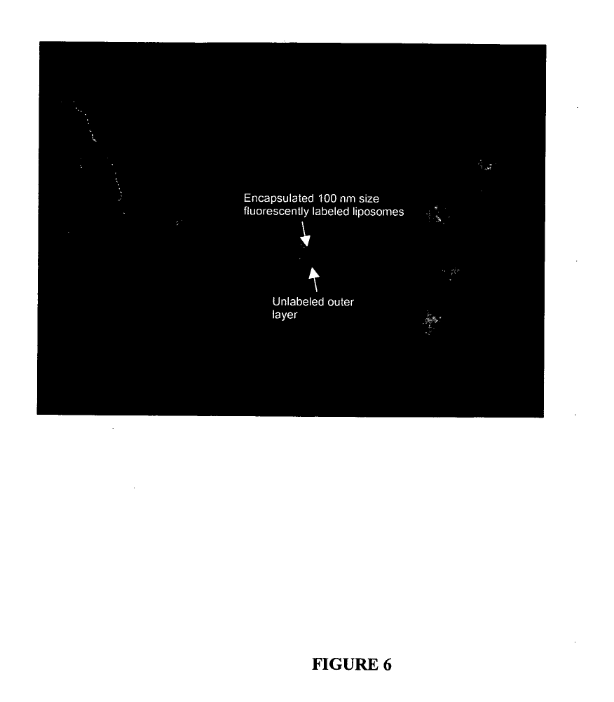

For example, rhodamine-DHPE labeled secondary liposomes were extruded to a diameter of 100 nm and encapsulated by larger primary liposomes in accordance with Example II. See, FIG. 6. The unencapsulated 100 nm diameter rhodamine-DHPE labeled liposomes were sufficiently eliminated by large-pore dialysis. Observations show that the encapsulated liposomes undergo Brownian motion but are not fused to one another.

Some embodiments contemplated by the present invention have numerous functional advantages over previous liposome compositions including, but not limited to: i) topologically complex liposome drug delivery systems can be prepared without any organic solvents; ii) topologically complex liposome drug delivery systems can be made with amphiphilic molecules, thereby creating hydrophilic surfaces and facilitating tissue adsorption and encapsulation (i.e., uptake); iii) topologically complex liposomes have increased surface stability thereby prolonging product shelf-life; iv) as opposed to oil suspensions, the surface properties of topologically complex liposome drug delivery systems can be easily modified by altering their lipid compositions; for example, to enhance the liposome stability or increase the tissue uptake rate; v) topologically complex liposome drug delivery systems can carry and deliver practically any drug (i.e., hydrophilic and/or lipophilic) that does not physically disrupt the liposome and/or chemically react with it could be encapsulated and delivered; vi) topologically complex drug delivery systems can be used to sequentially release different drugs wherein drugs within a secondary liposome population may undergo synchronized photodynamic release

Some embodiments contemplated by the present invention have the advantage to provide intracellular delivery of a drug and/or compound. In one embodiment, a topologically complex liposome population comprises a primary liposome population encapsulating a secondary liposome population, wherein the secondary liposome population has a fusogenic membrane layer. In one embodiment, the secondary liposome population encapsulates a drug or compound having an intracellular target. In one embodiment, the drug and/or compound comprises RNAi.

Some embodiments contemplated by the present invention have numerous technical advantages over previous liposome compositions including, but not limited to, combining the techniques of liposome extrusion and subsequent large pore dialysis to produce a uniform-sized topologically complex liposome population comprising a primary liposome population encapsulating a first drug and a second liposome population, wherein the second liposome population encapsulates a second drug. Using large pore dialysis to remove unencapsulated secondary liposomes to create a purified topologically complex liposome population has not previously been contemplated in the art.

### II. Uniform-sized Liposome Populations

Liposomes made by conventional methods that produce a population that is highly heterogeneous in diameter sizes. Knight et al., “Anticancer effect of 9-nitrocamptothecin liposome aerosol on human cancer xenografts in nude mice” Cancer Chemother Pharmacol 44: 177-186 (1999); Korgel et al., “Vesicle size distributions measured by flow field-flow fractionation coupled with multiangle light scattering” Biophysical Journal 74:3264-3272 (1998).

Various attempts have been made to produce uniform-size liposomes, including: i) extrusion (U.S. Pat. No. 5,008,050; herein incorporated by reference)); ii) gel filtration (Enoch, et al., “Formation and Properties of 1000-A-Diameter, Single-Bilayer Phospholipid Vesicles” Proc Natl Acad Sci USA 76:145-149 (1979)); iii) high performance size exclusion chromatography (HPSEC) (Grabielle-Madelmont et al., “Characterization of loaded liposomes by size exclusion chromatography” J Biochem Biophys Meth 56:189-217 (2003)); and iv) double emulsion (Utada et al., “Monodisperse double emulsions generated from a microcapillary device” Science 308:537-541 (2005); and Lorenceau et al., “Generation of polymerosomes from double-emulsions” Langmuir 21:9183-9186 (2005)).

Extrusion techniques have been used as a method for making liposomes in sizes ranging from 50 nm to 100 nm. Olson et al., “Preparation of Liposomes of Defined Size Distribution by Extrusion through Polycarbonate Membranes” Biochim Biophys Acta 557:9-23 (1979); and Hope et al., “Production of Large Unilamellar Vesicles by a Rapid Extrusion Procedure—Characterization of Size Distribution, Trapped Volume and Ability to Maintain a Membrane-Potential” Biochim Biophys Acta 812:55-65 (1985). Although it is not necessary to understand the mechanism of an invention, it is believed that extrusion forces liposomes to pass through membrane pores smaller than their sizes, such that they break down into smaller ones close to the pore sizes. Patty et al., “The pressure-dependence of the size of extruded vesicles” Biophysical J 85:996-1004 (2003). Obviously, extrusion does not eliminate liposomes smaller than the membrane pores, thereby resulting in a loss of efficacy when large liposomes are needed (i.e., for example, >200 nm). It is possible that the polydispersity of these extruded liposomes has been underestimated because the techniques used to analyze liposome sizes in these studies (multiangle laser light scattering), are reported to be inefficient when measuring polydisperse size distributions. Korgel et al., “Vesicle size distributions measured by flow field-flow fractionation coupled with multiangle light scattering” Biophysical J74:3264-3272 (1998).

Gel filtration techniques (i.e., for example, HPSEC in conjunction with extrusion) have also been employed to create uniform-sized liposome populations. Korgel et al., “Vesicle size distributions measured by flow field-flow fractionation coupled with multiangle light scattering” Biophysical J74:3264-3272 (1998); and Grabielle-Madelmont et al., “Characterization of loaded liposomes by size exclusion chromatography” J Biochem Biophys Meth 56:189-217 (2003). However, a major disadvantage of this technique is that the liposome average size diameter is limited to only 300 nm; the pore sizes of commercially available sieving gels. Consequently, it becomes increasingly difficult for current techniques to make uniform-size liposomes from 300 nm to several microns. Further disadvantages include, but are not limited to, a great deal of time and effort to run a large column that produces only a small amount of uniform-size liposomes, thereby making industrial-scale use impractical.

The double emulsion technique has been used to make uniform-size giant polymersomes (i.e., for example, 10 microns to several hundred microns) using a microfluidic device. Utada et al., “Monodisperse double emulsions generated from a microcapillary device” Science 308:537-541 (2005); and Lorenceau et al., “Generation of polymerosomes from double-emulsions” Langmuir 21:9183-9186 (2005). However, double emulsions have numerous disadvantages, including, but not limited to: i) the polymersomes are too large for most drug delivery applications; ii) the technique is constrained to microfluidic device scales; iii) the liposome compositions used are limited to certain membrane-forming polymers and phospholipids; and iv) the organic solvent contamination is still an unsolved issue except for making certain polymersomes. Several attempts have been made to make uniform-size liposomes by varying the concentration of organic solvent in a lipid-water solution. U.S. Pat. No. 5,049,388; and U.S. Pat. No. 6,596,305 (both herein incorporated by reference). Nevertheless, the resultant liposomal populations are still polydisperse by current standards. Furthermore, the size analysis instruments used in these studies (dynamic light scattering and submicron particle sizer) are primarily designed for the express purpose of measuring relatively uniform-size particles; therefore, polydisperse particles would appear uniform in size if measured by these instruments.

In one embodiment, the present invention contemplates a liposome-within-a-liposome drug delivery system created by producing uniform-sized liposomes utilizing an extrusion process followed by dialysis. The dialysis technique utilizes commercially available large-pore dialysis membranes to effectively drive the removal of both unencapsulated drug and liposomes having an average diameter that is less than the pore size of the dialysis membrane. Dialysis sets a lower limit for overall liposome average diameter. On the other hand, the initial extrusion process sets the upper limit for overall liposome average diameter. Consequently, when extrusion and dialysis are used in combination, a narrow range of uniform-sized liposomes may be created. As such, the difference between the pore size diameters between the initial extrusion process and the dialysis membranes determines the size range of the liposomes. As the extrusion-dialysis pore size difference minimizes, the liposome population average diameter becomes more uniform (i.e., for example, the variability would decrease as measured by either conventional variance measures and/or standard error of the mean).

Making liposomes uniform in size improves both drug delivery efficacy and safety. Conventional extrusion methods are limited to creating liposomes that are smaller than a specified size, thereby resulting in a polydisperse size distribution. Techniques using polycarbonate-membrane-based large-pore dialysis eliminate liposomes smaller than the pores of dialysis membranes. Uniform-sized liposome populations can be made using the combination of extrusion with large-pore dialysis. The efficacy, efficiency, and procedural simplicity of this technique make it useful for industrial applications. For example, when treating a cancerous tumor, small liposomes will enter a tumor capillary preferentially over a normal tissue capillary. However, if the liposome is too small, the liposome will not be trapped within the tumor to deliver the therapeutic drug. Consequently, a population of uniform-sized liposomes can maximize effective dose delivered to a tumor target.

Dialysis techniques were initially developed to remove detergent from detergent—lipid aggregates in solution, and then used to form primary liposome populations. Milsmann et al., Preparation of Large Single Bilayer Liposomes by a Fast and Controlled Dialysis” Biochim Biophys Acta 512:147-155 (1978); and Schubert, R., “Liposome preparation by detergent removal” Liposomes, Pt A 367:46-70 (2003). In one embodiment, the present invention contemplates a method of large-pore dialysis comprising polycarbonate track-etched membranes, wherein the membranes are capable of removing liposomes smaller than the membrane pores. Although it is not necessary to understand the mechanism of an invention, it is believed that when following extrusion, which sets an upper limit to the sizes, dialysis produces a liposome population within a narrow, and predictable, size range. Compared to the conventional techniques of making uniform-size liposomes, this technique can be applied to various kinds of liposomes, no matter what method or lipid compositions were used to make them; it can handle sizes at a wider range, especially the sizes useful for drug delivery; and the procedural simplicity of this technique makes it promising for industrial-scale applications.

### II. Differentially Sized Liposomes

In one embodiment, the present invention contemplates compositions and methods for improving existing liposomal multi-drug delivery systems. In one embodiment, a method comprises encapsulating a series of differentially sized liposomes within a liposomal multi-drug delivery system, wherein a larger liposome population encapsulates one- or more populations of smaller liposome populations. Although it is not necessary to understand the mechanism of an invention, it is believed that these compositions can deliver almost any drug (i.e., hydrophobic and hydrophilic), wherein delivery of a different drug to separate tissue targets and at different times can be controlled by lipid bilayer composition and/or the average size diameter of the smaller liposome population. Further, it is believed that the presently disclosed liposomal multi-drug delivery system segregates multiple drugs by encapsulating a “liposome-within-a-liposome”, which prevents aggregation and/or reaction between the drugs. In one embodiment, each “liposome-within-a-liposome” comprises a separate entity and respective lipid, bilayer membranes between each respective liposome do not interact with the other liposome.

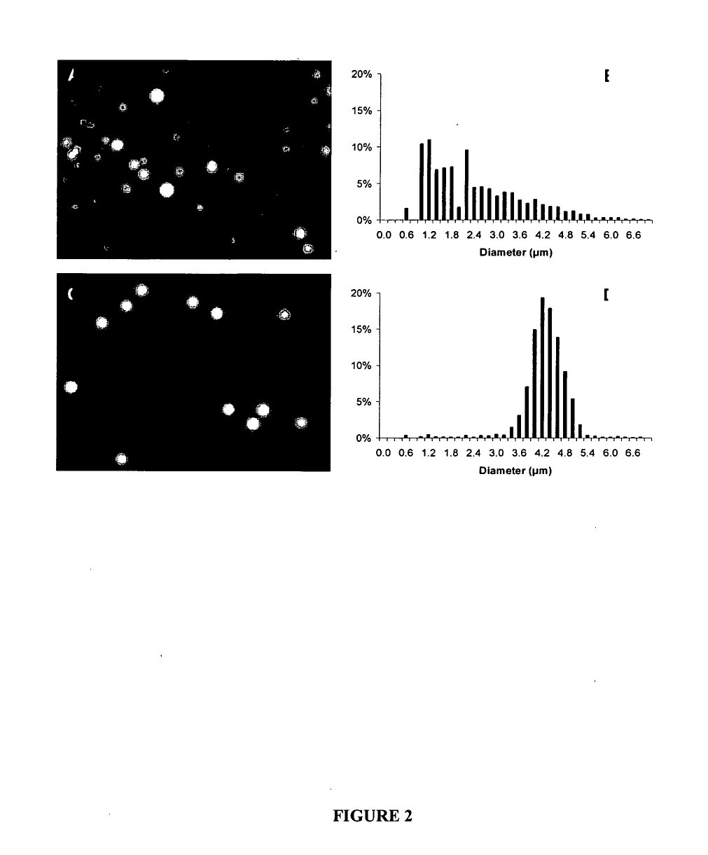

Oleic acid liposomes produced according to Example I were extruded with a 5-μm-pore-size membrane, were mostly less than 5 μm in diameter, but were still highly polydisperse. See, FIG. 2A. The corresponding histogram representations of the average size distributions are shown. See, FIG. 2B. Liposomes in the size range between 0˜0.5 μm, though believed to be present, could not be seen due to the limitation of optical microscopy at the magnification used. After 12 rounds of dialysis with 3-μm-pore-size membranes, liposomes smaller than 3 μm in the population were significantly reduced, resulting in a narrow size distribution between 3˜5 μm. See, FIGS. 2C & 2D. Statistical analysis suggested that the average diameter was 4.2 μm, with a standard deviation of 15%. Moreover, the concentration of these dialyzed liposomes was not significantly lowered compared to the extruded liposomes, making it suitable for high-concentration drug storage and administration.

Although it is not necessary to understand the mechanism of an invention, it is believed that if lipids are in solution at a concentration higher than the critical aggregation concentration, the washing buffer will inevitably contain spontaneously formed liposomes. Consequently, using a washing buffer would exchange dye/drug-encapsulating liposomes comprising a smaller average diameter than the membrane pores of the dialysis cassette. The resultant liposome population contains a uniform-sized dye/drug-encapsulating liposome population and a smaller liposome population that is dye and/or drug-free, thereby creating a “pseudo-monodispersity”. This “pseudo-monodispersity” preparation is equally as effective as preparation where all spontaneously formed drug-free liposomes were eliminated. These smaller drug-free liposomes would either not cause toxicity to normal tissues or since they cannot be seen, would not affect most research results. It is further believed that “pseudo-monodisperse” preparations might actually improve the long-term stability of the uniform-size dye/drug-encapsulating liposome populations. On the other hand, if the lipid concentration in the washing buffer is at, or below, its critical aggregation concentration, very few liposomes will be spontaneously formed in the washing buffer. Therefore, a dialyzed uniform-size liposome population formed under these conditions should contain a negligible amount of smaller dye and/or drug-free liposomes.

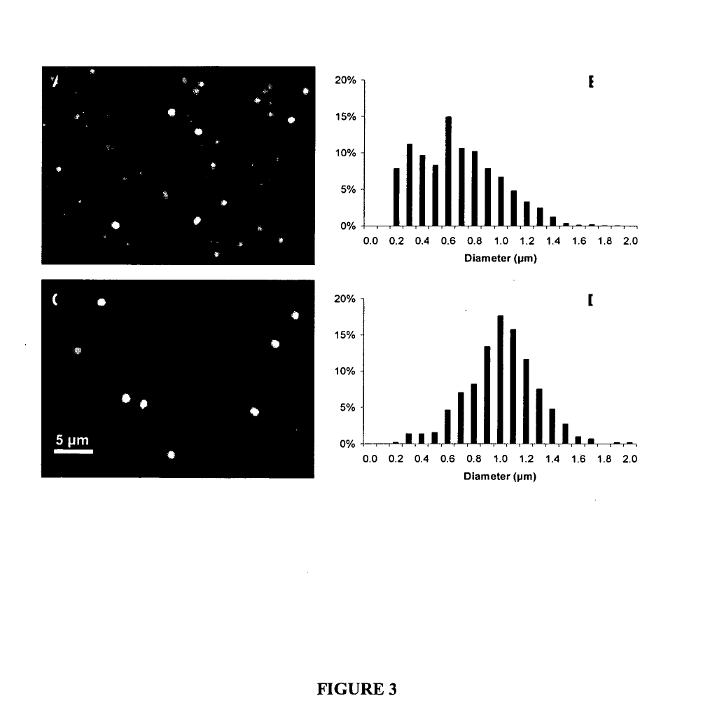

1-Palmitoyl-2-Oleoyl-sn-Glycero-3-Phosphocholine (POPC) can be used as a lipid for forming liposome populations to support a drug delivery system. Example I provides procedures for making uniform-sized POPC liposome populations. Dye-encapsulating POPC liposomes were extruded with a 1 μm-pore-size membrane thereby forming a polydisperse liposome population of less than 1 μm. See, FIGS. 3A & 3B. After dialyzing with 0.8-μm-pore-size membranes for 12 rounds, the population was significantly reduced in liposomes having an average diameter of smaller than 0.8 μm. See, FIGS. 3C & 3D. The analyzed average diameter was 1.0 μm with a standard deviation of 30%. It is notable that due to the limitations of optical microscopy and analysis software, particularly for particles smaller than 1 μm, the size distribution measured could appear more polydisperse than in the real case. For example, in a control experiment, uniform-size beads were analyzed by the same technique that presented a wider size distribution than that provided by the manufacturer (data not shown).

Although it is not necessary to understand the mechanism of an invention, it is believed that liposomes made by any method and of any lipid composition could be converted into a uniform-sized liposome population by combining extrusion and large-pore dialysis, thereby making this technique generic. For example, 300-400 μl of a highly concentrated uniform-size liposome sample has been produced by extrusion and large-pore dialysis, making this technique more efficient and cost-effective than any other current lab techniques. Furthermore, the simplicity of this technique allows it to be commercially applicable. For example, industrial-scale dialysis could be achieved through making dialysis flow channels. In the United States Food & Drug Administration's publication Liposome Drug Products “particle size (mean and distribution profile)” and “volume of entrapment in liposomal vesicles” are listed as the physicochemical properties critical for liposome products. However, because a commercially applicable technique to make liposomes uniform in size was not available, no practical standards concerning these properties were set for the industry at the time. This technique can also be used as a cost-effective way of making uniform-size solid micro/nano particles, which are widely used in drug delivery and biomedical research.

Compared to the above discussed nanoparticle-dependent liposomal drug delivery systems, some embodiments of the present invention have little limitation on the physical and chemical properties of the drugs capable of being delivered. Although it is not necessary to understand the mechanism of an invention, it is believed that any drug that does not physically disrupt, or chemically react with, the liposome membrane structure can be encapsulated and delivered.

In one embodiment, the present invention contemplates a topologically complex liposome comprising at least two drugs, wherein each drug is separated from the other by at least one lipid bilayer. In one embodiment, the lipid bilayer encapsulates an aqueous environment. In one embodiment, the lipid bilayer encapsulates a lipid environment. In one embodiment, the separation prevents drug aggregation and/or crystallization. In one embodiment, the separation prevents a chemical reaction between the two drugs. In one embodiment, the separation prevents drug degradation, whether due to enzymes (i.e., for example, proteases and/or nucleases) or other environmental instabilities that limits shelf-life (i.e., for example, temperature, sunlight etc.). In one embodiment, the drugs comprise water-soluble drugs. In one embodiment, the drugs comprise lipid-soluble drugs. In one embodiment, the drugs reside within the encapsulated environment. In one embodiment, the drugs reside within the lipid bilayer membrane.

In one embodiment, the present invention contemplates a topologically complex liposome comprising at least four drugs. In one embodiment, a first drug is encapsulated by a secondary liposome bilayer membrane. In one embodiment, a second drug is encapsulated by a primary liposome bilayer membrane. In one embodiment, a third drug is embedded within the secondary liposome bilayer membrane. In one embodiment, a fourth drug is embedded within the primary liposome bilayer membrane. In one embodiment, the second drug comprises a photosensitizer.

### III. Liposomes Having Differential Lipid Compositions

In one embodiment, the present invention contemplates a uniform-sized topologically complex liposome comprising a primary liposome population encapsulating a secondary liposome population. In one embodiment, the primary liposome population comprises a membrane lipid different from the secondary liposome population. In one embodiment, the primary liposome population comprises a pH-sensitive phospholipid and the secondary liposome population does not comprise a pH-sensitive phospholipid. In one embodiment, the primary liposome population comprises a non-fusogenic phospholipid and the secondary liposome population comprises a fusogenic phospholipid.

A. pH-Sensitive Phospholipids

Some phospholipid compositions are reported to be pH-sensitive meaning that they can get lysed when there is a pH drop, either physiologically moving from one environment to another, or through light-triggered internal pH-drop.

pH-sensitive phospholipids are useful in creating pH-sensitive liposomes. It is believed that pH-sensitive liposomes release a larger proportion of their contents than do non-pH sensitive liposomes when the pH of the surrounding environment decreases from physiological pH (pH 7.4) to a pH between about 3.5 and 6.5. This characteristic can be exploited to advantage for in vitro and in vivo uses. Liposomes may be internalized by cells via the endocytic pathway, and are exposed in the endosome to a decreasing pH. Since liposomes can be targeted to cells using appropriate ligands such as antibodies directed to epitopes on cells of interest, a liposome stable at pH 7.4 but unstable at a pH encountered in the endosome can be used to deliver its contents preferentially to the targeted cell population. Further, since many tumors tend to have a pH of between 5.8 to 6.5, liposomes stable at pH 7.4, but destabilized at a pH of 5.8-6.5, can be used to deliver cytotoxic agents, imaging agents, or other desired agents preferentially to tumor cells. In some embodiments, the present invention contemplates using topologically complex pH-sensitive liposome populations comprising tumor targeting agents, wherein the tumors are more acidic than surrounding normal tissue. In one embodiment, the tumor has a pH of about 6.5 or less, and even more preferably those with a pH of about 6 or less. Papahadjopoulos et al., “pH-Sensitive, Serum-Stable Liposomes”, U.S. Pat. No. 6,426,086 (herein incorporated by reference).

In one embodiment, the present invention contemplates a pH sensitive primary liposome. Although it is not necessary to understand the mechanism of an invention, it is believed that one pathway for the entry of liposomes into cellular cytoplasm is by endocytosis into lysozymes of low pH. In one embodiment, the present invention contemplates a pH sensitive primary liposome having stability at neutral pH but lyse at an acidic pH. In one embodiment, the liposome can be used to deliver enzymes into the lysozymes of the cytoplasm, whereupon the contents are released upon lysis.

In one embodiment, liposomes can be made sensitive to low pH by modifying the lipid composition. For example, pH sensitive liposomes can be prepared by using phospholipids which form lipid bilayers when charged, but fail to stack in an ordered fashion when neutralized. An example of such a phospholipid is phosphatidylethanolamine, which is negatively charged above pH 9. The net charge of a phospholipid can be maintained at a pH which would otherwise neutralize the head groups by including charged molecules in the lipid bilayer which themselves can become neutralized. Examples of these charged molecules are oleic acid and cholesteryl hemisuccinate, which are negatively charged at neutral pH but become neutralized at pH 5. The effect of combining these together in a lipid bilayer is that at pH 9 all molecules are charged; at pH 7 the net negative charge of the oleic acid and cholesteryl hemisuccinate maintains the stability of the phosphatidylethanolamine, and at pH 5 all components are protonated and the lipid membrane is destabilized. Additional neutral molecules, such as phosphatidylcholine, can be added to the liposomes as long as they do not interfere with stabilization of the pH sensitive phospholipid by the charged molecules.

In other embodiments pH sensitive liposomes may be fused when their medium is treated to make the pH acidic. These liposomes become pH sensitive when greater than about 20 mol percent of an amphipathic molecule containing one or more weakly acidic functional groups, such as the carboxylic group. Compounds of this type include palmitoylhomo-cysteine and long chain, i.e., C₁₂ to C₃₀, preferably C₁₆ to C₂₄, fatty acids such as palmitic acid and oleic acid. The presence of an amphipathic molecule which has a tendency to form hexagonal phase or inverted micelles, such as phosphatidylethanolamine, greatly enhances this fusion process. In one demonstrated example, infra, a preferred mole ratio of phosphatidylethanolamine to palmitoyl homocysteine is 8:2.

Although it is not necessary to understand the mechanism of an invention, it is believed that the lipid composition of a liposome strongly determines the efficiency of the acid induced fusion. Liposome fusion facilitated at pH 4.8 is optimal in the presence of palmitoyl homocysteine. However, combinations of phosphatidylethanolamine and palmitoyl homocysteine (i.e., for example, at a molar ratio of approximately 8:2) facilitated effective fusion, whereas the addition of phosphatidylcholine to such liposomes diminished fusion.

In addition to phosphatidylethanolamine and palmitoyl homocysteine liposome compositions, other lipids may also provide pH sensitive liposomes. For example, liposomes containing phosphatidylethanolamine and palmitic acid (i.e., for example, at a molar ratio of approximately 8:2) result in effective liposome fusion at about pH 6.3 and below. Further, liposomes containing phosphatidylethanolamine and oleic acid (8:2) demonstrated a rapid fusion rate at about pH 6.3 and below.

B. Fusogenic Phospholipids

Some phospholipids are reported to be fusogenic, or alternatively, non-fusogenic. In general, a fusogenic phospholipid facilitates and stimulates membrane fusion, while a non-fusogenic phospholipid minimizes membrane interactions, thereby preventing membrane fusion.

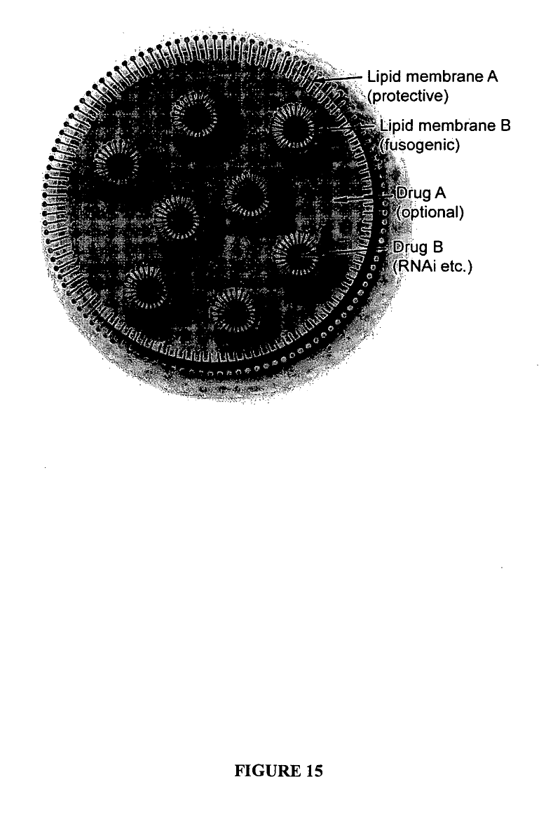

In one embodiment, the present invention contemplates a topologically complex liposome population comprising a primary liposome encapsulating a secondary liposome. The primary liposome may have a lipid membrane composed of non-pH sensitive, stable, and anti-fusogenic lipid compositions, thereby acting as a protective layer to extend the circulating time of the liposome. See, FIG. 15 (lipid membrane A). The secondary lipid membrane have fusogenic lipid compositions, thereby enhancing the fusion or endocytosis of the liposome to target cells. See, FIG. 15 (lipid membrane B). The aqueous membrane compartment of the primary liposome may, or may not contain a drug or other substance, and/or a variety of compounds to enhance the localized release and/or cell-uptake of the secondary liposomes. See FIG. 15 (Drug A). The aqueous membrane compartment of the secondary liposome may, or may not, contain a drug or other substance (i.e., for example, an RNAi) to be delivered intracellularly. See, FIG. 15 (Drug B).

The encapsulation of a fusogenic liposome with a non-fusogenic liposome solves a problem posed in the art, such as:
“Many pharmaceutical agents, including various large molecules (proteins, enzymes, antibodies) and even drug-loaded pharmaceutical nanocarriers, need to be delivered intracellularly to exert their therapeutic action inside cytoplasm or onto nucleus or other specific organelles”
   Torchilin, V. P., “Recent approaches to intracellular delivery of drugs and DNA and organelle targeting” Annu Rev Biomed Eng 8:343-375 (2006),
   and.
“Effective delivery is the most challenging hurdle remaining in the development of RNAi as a broad therapeutic platform”, as “systemic delivery of siRNA to target tissues deep within the body remains challenging.”. The two major obstacles in RNAi delivery in vivo are “promote cellular uptake and the release of the drug into the cytoplasm” and “proper pharmacokinetics” (long circulating time).
   de Fougerolles, A., et al., “Interfering with disease: a progress report on siRNA-based therapeutics” Nat Rev Drug Discov, 6:443-453 (2007).

Fusogenic liposomes can be produced from a fusogenic lipid (i.e., for example, DOPE). Such fusogenic lipids enhance: i) cell uptake; ii) electrostatic/hydrophobic interaction between liposomes and cell membranes, and iii) liposome attachment to cell surface, fusion, and/or endocytosis, thereby facilitating the delivery of certain incorporated molecules (i.e., for example, ligands, antibodies, viral antigens, aptamers, etc.). U.S. Pat. Nos. 5,891,468 and 7,108,863 (both herein incorporated by reference). Such viral antigens may include, but are not limited to, in SFV spike protein and influenza virus hemagglutinin, that can facilitate liposome fusion into certain cell types. Hosaka et al., “Hemolysis by liposomes containing influenza virus hemagglutinins” J Virol 46:1014-1017 (1983); and Maeda et al., “Interaction of influenza virus hemagglutinin with target membrane lipids is a key step in virus-induced hemolysis and fusion at pH 5.2” Proc Natl Acad Sci USA, 78:4133-4137 (1981).

Unfortunately, liposomes having an improved ability for fusion and/or or endocytosis into target cells would certainly be more easily uptaken by macrophages and quickly cleared from bloodstream before reaching the target tissue. Alternatively, liposomal surface modifications intended to extend circulating time may compromise their fusogenic ability. To resolve this problem, the present invention contemplates a method for making topologically complex liposome to produce liposomes that possess both fusogenic and long-circulating properties.

In one embodiment, the present invention contemplates a fusogenic, long-circulating topologically complex liposome. See, FIG. 15. The illustrated primary liposome (lipid membrane A) is composed of non-pH sensitive, stable, and anti-fusogenic lipid compositions. Such lipid compositions may include, but are not limited to, 60 mol % POPC 30 mol % cholesterol and 10 mol % PEG/PEG lipid. Lipid membrane A may also contain ligands, antibodies, and aptamers (which can be different from the ones on lipid membrane B that interact with the target cells) that target specific receptors on the endothelium cells on the vascular wall in the target organ. Such lipid compositions help to extend the circulating time of the liposome: POPC is non-pH and stable in serum, and cholesterol helps to stabilize the formulation; PEG/PEG lipid helps to avoiding immune recognition and endocytosis in bloodstream. The illustrated secondary liposomes (lipid membrane B) are composed of fusogenic lipid compositions. Such lipid compositions may include, but are not limited to, combinations of cationic and fusogenic lipids, viral antigens, ligands, antibodies, and aptamers. Such lipid compositions enhance the fusion or endocytosis of liposome to the target cells: cationic lipids (i.e., DOPE) are positively charged lipids and can interact with cell membranes; viral antigens (i.e., SFV spike protein and influenza virus hemagglutinin) can facilitate the liposome fusion into certain cell types); ligands, antibodies, and aptamers to target cell receptors can mediate liposome attachment to cells.

The figure also exemplifies Drug A as encapsulated by the primary liposome, and is optional to have in this invention. Such drug composition may include, but are not limited to, a photosensitizer and a oxidation substrate (i.e., HPTS and bicine) that induced light-activated localized release of the secondary liposomes, a drug that stimulates the target cells to uptake the secondary liposomes, and a vasodilator drug that increases the blood vessel permeability and helps the secondary liposomes to enter the target tissue from blood circulation.

The figure also exemplifies Drug B as encapsulated by the secondary liposomes, and the drug can be delivered intracellularly. Such drugs may include, but are not limited to, RNA molecules (i.e., RNAi), DNA molecules, large-molecule drugs (i.e., proteins), high-systemic-toxicity drugs, and drugs that have short lifetime in blood circulation.

In one embodiment, the present invention contemplates a method for making a topologically complex lipsome population comprising a non-fusogenic primary liposome population and fusogenic secondary liposome population including, but not limited to: DOPE (1,2-dioleoyl-sn-glycero-3-phosphoethanolamine), POPC (1-palmitoyl-2-oleoyl-sn-glycero-3-phosphocholine), and DSPE-PEG (distearoylphosphatidylethanolamine-polyethyleneglycol) are obtained from Avanti Polar Lipids (Birmingham, Ala.). The purification of influenza virus hemagglutinin is described. Hosaka et al., “Hemolysis by liposomes containing influenza virus hemagglutinins” J Virol 46:1014-1017 (1983).

DOPE is codissolved with 3% (w/w) influenza virus hemagglutinin in methanol solution. The organic solvents were completely removed by rotary evaporation overnight. The lipid sample was resuspended by a drug B solution (in PBS buffer at pH 7.4), then vortexed briefly, and tumbled overnight. These liposomes were extruded 11 passes by a stack of two Nuclepore® polycarbonate track-etched membranes with 100 nm diameter pores, using the Mini-extruder system (Avanti Polar Lipids, Inc.). Olson et al., “Preparation of Liposomes of Defined Size Distribution by Extrusion through Polycarbonate Membranes” Biochimica Et Biophysica Acta 557:9-23 (1979); Hope et al., “Production of Large Unilamellar Vesicles by a Rapid Extrusion Procedure—Characterization of Size Distribution, Trapped Volume and Ability to Maintain a Membrane-Potential” Biochimica Et Biophysica Acta 812:55-65 (1985); Hanczyc et al., “Experimental models of primitive cellular compartments: Encapsulation, growth, and division” Science 302:618-622 (2003). The extruded secondary liposomes were dialyzed using a 10 k MW regular (as opposed to large-pore dialysis as used later) dialysis cassette, in PBS buffer at pH 7.4 to remove unencapsulated drug B. Drug A may be added at this step depending on the application.

The sample is then added into a lipid mixture of POPC:cholesterol:DSPE-PEG (2:1:0.2 mol), vortexed and tumbled briefly, allowing the primary liposomes to form. The sample is dialyzed by PBS buffer at pH 7.4. The 3-μm-pore-size cassette used in dialysis is modified from commercially available 500 μl dialysis cassettes (Pierce Biotechnology, Inc.). 30 ml of washing buffer is used in each round of dialysis, which just submerges the laid-down dialysis cassette in a 150 ml beaker, with shaker speed set to 60 rpm. The first 5 rounds of dialysis takes 5˜10 min each, and 7 additional rounds each at 2 hours minimum are done to sufficiently eliminate the secondary liposomes unencapsulated by the primary liposomes.

### IV. Blood Substitutes

In one embodiment, the present invention contemplates a uniform-sized liposome population encapsulating blood substitutes. In one embodiment, the blood substitute comprises hemoglobin.

General classes of blood substitute products currently under development include, but are not limited to: i) surface-modified or polymerized hemoglobin; ii) perflorocarbons; and iii) liposome-encapsulated hemoglobin. Winslow, R. M., “New transfusion strategies: red cell substitutes” Annu Rev Med, 50: 337-353 (1999); Stowell, C. P., “Hemoglobin-based oxygen carriers” Curr Opin Hematol 9: 537-543 (2002); U.S. Pat. Nos. 4,911,929, 5,049,391, and 5,670,173 (all herein incorporated by reference). Among them, polymerized hemoglobin products are under Phase III clinical trials (i.e., for example, Hemopure®, Biopure Corp; and PolyHeme®, Northfield Laboratories). U.S. Pat. No. 7,135,554. (herein incorporated by reference).

Of those available, liposome-encapsulated hemoglobin are reported to be “most like native red cells” and thus is considered the most promising product as a blood substitute. Winslow, R. M., “New transfusion strategies: red cell substitutes” Annu Rev Med, 50: 337-353 (1999). However, those in the art have identified that:
“the development of liposome-encapsulated hemoglobin as a blood substitute has lagged behind the other two approaches”
   as,
“the technology for reproducibly making liposomes of a uniform size distribution is complex and is at least partly responsible for the slower progress with these substitutes”
   Stowell, C. P., “Hemoglobin-based oxygen carriers” Curr Opin Hematol 9: 537-543 (2002). In particular, their size “dictates important parameters such as circulation persistence and organ biodistribution” Rudolph, A. S., “Biomaterial Biotechnology using self-assembled lipid microstructures” J Cell Biochem 56: 183-187 (1994).

In one embodiment, the present invention contemplates a solution to these problems by a method for making uniform-sized liposomes to produce uniform-sized liposomes encapsulating hemoglobin to be used as a blood substitute. In one embodiment, the method includes, but is not limited to: POPC (1-palmitoyl-2-oleoyl-sn-glycero-3-phosphocholine), cholesterol, and DSPE-PEG (distearoylphosphatidylethanolamine-polyethyleneglycol) obtained from Avanti Polar Lipids (Birmingham, Ala.). Hemoglobin (Hb) (either from human, animal, or recombinant) is obtained from Sigma (Sigma-Aldrich, St. Louis, Mo.). The encapsulated purified Hb (38 g/dL) contained 14.7 mM pyridoxal 5′-phosphate (PLP, Sigma-Aldrich, St. Louis, Mo.) as an allosteric effector at a molar ratio of PLP/Hb) 2.5. Recombinant human serum albumin can be obtained from Nipro Corp. (Osaka, Japan).

A lipid mixture of POPC:cholesterol:DSPE-PEG (2:1:0.2 mol) is prepared in a methanol/chloroform solution. The organic solvents are completely removed by rotary evaporation overnight. The lipid sample is resuspended in the Hb solution, then vortexed briefly, and tumbled overnight. These liposomes are extruded 11 passes by a stack of two Nuclepore® polycarbonate track-etched membranes with 1 μm diameter pores, using the Mini-extruder system (Avanti Polar Lipids, Inc.). Olson et al., “Preparation of Liposomes of Defined Size Distribution by Extrusion through Polycarbonate Membranes” Biochimica Et Biophysica Acta 557:9-23 (1979); Hope et al., “Production of Large Unilamellar Vesicles by a Rapid Extrusion Procedure—Characterization of Size Distribution, Trapped Volume and Ability to Maintain a Membrane-Potential” Biochimica Et Biophysica Acta, 81255-65 (1985); and Hanczyc et al., “Experimental models of primitive cellular compartments: Encapsulation, growth, and division” Science 302:618-622 (2003). The sample is dialyzed with PBS buffer at pH 7.4. The 0.8-μm-pore-size cassette used in dialysis is modified from commercially available 500 μl dialysis cassettes (Pierce Biotechnology, Inc.). 30 ml of washing buffer is used in each round of dialysis, which just submerges the laid-down dialysis cassette in a 150 ml beaker, with shaker speed set to 60 rpm. The first 5 rounds of dialysis takes 5˜10 min each, and 7 additional rounds each at 2 hours minimum are done to sufficiently eliminate the unencapsulated Hb and the liposomes smaller than 0.8 μm. Recombinant human serum albumin is added to the dialyzed liposomes sample at 5 wt %. All procedures are done under sterile conditions.

### V. Photodynamic Liposomal Drug Release

A. Conventional Photodynamic Methods

Liposome-mediated drug delivery has been attempted for many different medical conditions. Each medical condition has a different set of parameters which liposomes encounter, thereby resulting in an empirical approach to identify successful techniques. For example, one major question in treating cancer is how to discriminate and destroy cancer cells while sparing normal cells. Photodynamic therapy has been under development for over 30 years in an attempt to provide localized cancer treatment. Dolmans et al., “Photodynamic therapy for cancer” Nat Rev Cancer 3:380-387 (2003). Although it is not necessary to understand the mechanism of an invention, it is believed that many photodynamic therapies are based on a central mechanism: certain chemicals (photosensitizers), under the activation of light, generate cytotoxic reactive oxygen species (ROS). These reactive oxygen species can trigger tumor necrosis by directly killing tumor cells, damaging tumor vasculature, and provoking immune responses. Both the selective uptake of the photosensitizer by the tumor and the photoactivation help to localize the generation of reactive oxygen species and reduce the damage to the surrounding tissue.

Nevertheless, despite all these advantages, the current therapeutic applications of photodynamic therapy are limited to the treatment of certain superficial, small, and early-stage tumors. Dolmans et al., “Photodynamic therapy for cancer” Nat Rev Cancer 3:380-387 (2003). As a result, conventional photodynamic therapy remains a cancer treatment of limited effectiveness for a number of reasons. First, most present-day photosensitizers have only a moderate absorption of light at the desired wavelengths (usually at infrared), and thus the tissue-damaging effect generated by a reactive oxygen species is limited. As a result, clinicians often resort high drug doses (thereby increasing side effects) and high illumination intensities to obtain clinically effective tissue-damaging effects. Second, reactive oxygen species generation is limited by the tissue oxygen supply, thereby requiring tissue re-oxygenization which reduces both the duration and intensity of illumination. Third, some tissues (i.e., for example, tumor vasculature) respond to injury by upregulating angiogenic factors such as vascular endothelial growth factor (VEGF) and cycloxygenase (COX-2) which, paradoxically, promote tumor growth. Ferrario et al., “Antiangiogenic treatment enhances photodynamic therapy responsiveness in a mouse mammary carcinoma” Cancer Res 60:4066-4069 (2000). Furthermore, as a side effect, patients often have to avoid sunlight exposure for 4-6 weeks after the treatment, due to the subcutaneous accumulation and low clearance rate of photosensitizers.

Targeted cancer therapy is used to identify and interfere with specific target molecules needed for carcinogenesis and tumor growth. Green, M. R., “Targeting targeted therapy” N Engl J Med 350:2191-2193 (2004). However, monoclonal antibodies are required to be generated having affinity for a specific target molecule, which is different for each type and/or subtype of cancer. This is a method which tends to be time-consuming and expensive. Moreover, targeting one particular pathway (by a single monoclonal antibody) is often not sufficient to kill the tumor because cancer cells can mutate and develop drug resistance. Therefore, targeted cancer therapies are generally more effective in treating early-stage cancers, within which not enough mutations have occurred for drug resistance to have developed. So far, they have been used for treating a few types of cancers (lymphoma, breast cancer, colon cancer, etc.) for which the molecular disorders are well understood and identifiable in the patient population.

Presently, the primary treatments for advanced-stage tumors remain surgery, radiation, and chemotherapy. Chemotherapy targets central cell replication pathways and has strong tumor suppression effects. One major problem is that chemotherapy tends to kill cancer cells and normal cells indiscriminately and causes serious side effects—even though cancer cells are fast-replicating, there are normal cells (hair cells, intestinal epithelium, bone marrow cells, etc.) in the body that are as active in replication. Clinically, a patient's tolerance of side effects has to be closely monitored, and these side effects are the key factors limiting the dosage and duration of the treatment.

B. Localized Photodynamic Drug Delivery

It would be ideal for a chemotherapeutic cancer treatment to be as localized as the conventional photodynamic therapy. It is believed that such a local delivery system would provide stronger tumor suppression effects. Various drug delivery systems, e.g., nanoparticle-based and liposomal chemotherapeutic drugs, have been developed in an attempt to localize drug release. Allen et al., “Drug delivery systems: entering the mainstream” Science 303:1818-1822 (2004); and Cheong et al., “A bacterial protein enhances the release and efficacy of liposomal cancer drugs” Science 314:1308-1311 (2006). There have also been attempts to use liposomes or nanoparticles for delivering photosensitizers to enhance the accumulation of photosensitizer in the tumor. Chen et al., “Liposomal delivery of photosensitising agents” Expert Opin Drug Deliv 2:477-487 (2005).

Modern imaging techniques are usually able to identify and locate most tumors, thereby allowing tumor location information to be integrated a part of the therapy design. For example, cancer surgeries often rely on tumor location information provided by imaging studies. Other surgical approaches, like the ‘gamma knife’ technique (developed to treat brain tumors) is less invasive. Nevertheless, the ‘gamma knife’ technique is limited small volume tumors. Ultrasound techniques have also been applied to lyse liposomes to provide localized drug release. Marmottant et al., “Controlled vesicle deformation and lysis by single oscillating bubbles” Nature 423:153-156 (2003). Even liposomes made of photosensitive lipids have been suggested to increase drug permeability during photoactivation. Bondurant et al., “Photoinitiated destabilization of sterically stabilized liposomes” Biochim Biophys Acta 1511:113-122 (2001). However, despite the increased permeability (˜200 fold) afforded by the photosensitive lipids, drug release profile is still slow (˜1 hr) compared to the timescale of blood flowing through the tumor. Consequently, liposomes comprising photosensitive lipids have difficulties in localizing drug release unless the liposomes are designed for rapid uptake by the tumor. Further, it is also uncertain whether liposomes comprising photosensitive lipids are permeable enough to release large-molecule drugs. Faster drug release from liposomes comprising photosensitive lipids has been attempted using a high-power pulse laser (˜10⁶ W). Bisby et al., “Photosensitive liposomes as ‘cages’ for laser-triggered solute delivery: the effect of bilayer cholesterol on kinetics of solute release” FEBS Lett 463:165-168 (1999). This approach, however, has the disadvantages in that high-power pulse lasers are very expensive and difficult to operate and have serious operational safety issues (i.e., for example, since these lasers are typically used for cutting metals they can cause severe tissue burns).

B. Synchronized Lysis of Topologically Complex Liposomes

The present invention contemplates a novel photodynamic therapy that overcomes limitations in the existing photodynamic methods or treatments. For example, conventional photodynamic therapy uses the direct tissue-damaging effects from the reactive oxygen species generated by photosensitizers to trigger tumor necrosis. In one embodiment, the present invention contemplates a method comprising photoactivated liposome lysis and local release of chemotherapeutic drugs. In one embodiment, liposomal lysis results in stronger tumor suppression effects because targeted drugs may be incorporated into the liposomes. In addition to treating cancer, virtually any drug can be encapsulated in the presently contemplated topologically complex liposomes.

In one embodiment, the present invention contemplates a uniform-sized primary liposome capable of synchronized photodynamic drug release. In one embodiment, the uniform-sized liposomes comprises a photosensitizer (i.e., for example, HPTS). In one embodiment, the liposomes are exposed to light, wherein the bicine is converted into an oxygen radical species (ROS), oxidizing bicine, and thereby producing a marked pH drop within the liposome. In one embodiment, the pH drop results in primary liposome lysis. In one embodiment, the primary liposome further comprises secondary liposome containing a therapeutic drug which are released upon the light exposure. Although it is not necessary to understand the mechanism of an invention, it is believed that the pH drop initiates an influx of sodium ion, thereby resulting in a synchronized liposomal lysis (i.e., less than 0.4 seconds). Alternatively, one can use pH-sensitive phospholipid to construct the primary liposome (U.S. Pat. No. 6,424,086 B1) (herein incorporated by reference), which, under light-triggered internal pH drop, will be lysed. Although it is not necessary to understand the mechanism of an invention, it is believed that the influx in sodium results in a high interliposomal osmotic pressure resulting in near simultaneous bursting of the vast majority of the targeted liposomes. In other embodiments, synchronized photodynamic vesicle release may occur from liposomes comprising either fatty acids, phospholipids or a combination of fatty acids and phospholipids. Although it is not necessary to understand the mechanism of an invention, it is believed that phospholipid vesicles are more stable than fatty acid vesicles under physiological conditions and are most commonly used for drug delivery.

This photodynamic osmotic-induced liposome drug release has many surprising advantages over conventional phototherapy. First, the contemplated photodynamic-induced liposomal therapy does not require the generation of large concentrations of ROS for the therapeutic effect. In contrast, a small amount of ROS is generated that triggers liposomal lysis and drug release. Consequently, the required concentration of the photosensitizer is much less, thereby reducing side effects. Second, the requirement for a lower overall concentration of ROS also results in a benefit of allowing reduced photoillumination intensity to initiate the process. Third, having the capability for liposomal lysis at lower photoillumination intensities allows the use of radiation sources, such has X-ray and gamma-ray. These radiation sources have the advantage of providing deeper tissue penetration, and the ability to provide a more focused beam for targeted therapy.

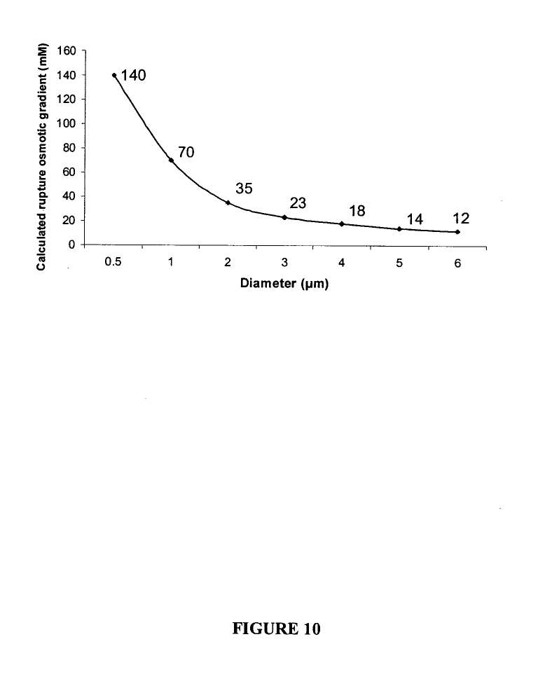

In one embodiment, the present invention contemplates a method of drug delivery comprising providing a uniform-sized liposome population comprising at least one drug created by a combined extrusion/dialysis technique that are capable of synchronized liposomal lysis following light illumination (i.e., for example, the liposome population ‘explodes’). In one embodiment, the method provides a locally administered bolus drug delivery. In one embodiment, the liposome lysis synchronization results from uniform size homogeneity Although it is not necessary to understand the mechanism of an invention, it was observed that in control experiments, where liposomes are heterogeneous, the lysis observed was not synchronized. (data not shown). In one embodiment, the liposomal lysis synchronization is size-dependent. See, FIG. 10.

The present invention contemplates that liposomes produced by any method, and of any lipid composition, can be processed by the extrusion/dialysis combination to produce topologically complex primary liposomes as described herein. In one embodiment, the method produces 300˜400 μl of a highly concentrated uniform-sized topologically complex primary liposome population. It is believed that this process is more efficient and cost-effective than any other current lab or industrial techniques. Furthermore, the simplicity of this technique is compatible with an expansion of scale for commercial application. For example, industrial-scale dialysis can be achieved through making dialysis flow channels. In addition to making uniform-sized liposomes, this technique can be adapted as a cost-effective way of making uniform-sized solid micro/nanoparticles, which are also widely used in drug delivery and biomedical research.

As describe herein, uniform-sized topologically complex primary liposomes can be created for controlled lysis induced by photoactivation and subsequent alterations in internal osmotic pressure. Applying Laplace's law, rupture osmotic pressure of oleic acid liposomes at given sizes was calculated. See, FIG. 10. This illustration shows that larger liposomes (i.e., for example, primary liposomes as contemplated herein) are more sensitive to the increase of internal osmotic pressure than smaller liposomes (i.e., for example, secondary liposomes as contemplated herein), which has been confirmed by experimental observations (data not shown). Therefore, even though light-controlled liposome lysis can also happen in a heterogeneous population of liposomes, only uniform-sized topologically complex primary liposomes undergo synchronized liposome lysis to precisely localize drug release.

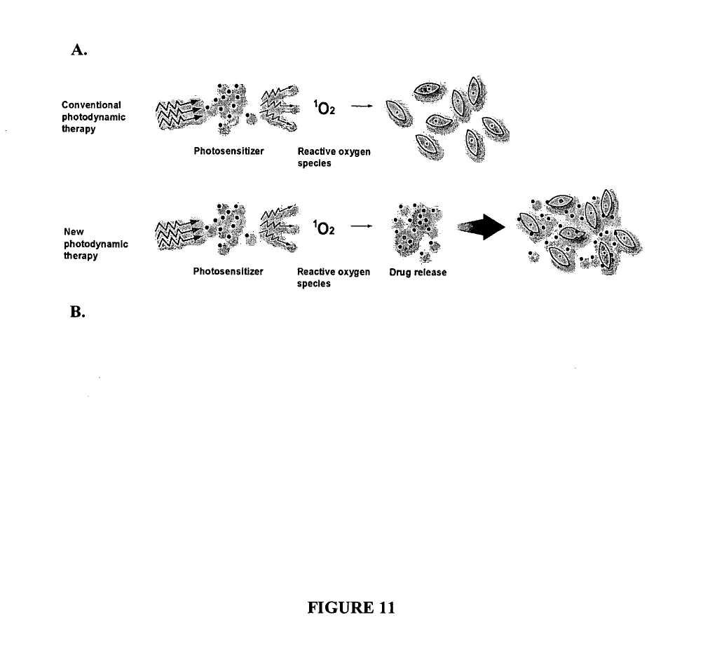

As discussed herein, conventional photodynamic therapy relies on the direct tissue-damaging effects from the reactive oxygen species generated by photosensitizers. However, these effects require a high concentration of photosensitizer, strong illumination, and sufficient oxygen supply to the tissue, greatly limiting the effectiveness of the treatment (in terms of treatable tumor volume and stage). See, FIG. 11A. In one embodiment, the present invention contemplates a photodynamic therapy method comprising less photosensitizer and less illumination intensity than conventional photodynamic therapy. Although it is not necessary to understand the mechanism of an invention, it is believed that less photosensitizer and illumination is necessary because the generated reactive oxygen species are only required to initiate a series of chemical/physical processes (i.e., for example, pH-induced Na⁺ influx), and not provide any therapeutic effectiveness (i.e., for example, tumor cell killing). In one embodiment, the present invention contemplates a topologically complex primary liposome population comprising a small amount of photosensitizer, wherein under a minimal illumination intensity, liposomal lysis occurs thereby releasing a therapeutically effective drug. See, FIG. 11B. As an analogy, a bullet cartridge is fired by using a small shock-sensitive primer to initiate the reaction, wherein only the gun powder, when ignited by the spark from the primer, creates sufficient explosive power to propel the bullet out of the cartridge. In one embodiment, a minimal illumination intensity is approximately 250 mW. It is believed that this minimal illumination intensity is safe for human tissues and does not require eye protection by either the subject or the operator. In one embodiment, swollen liposomes (i.e., for example, hypertonic) are lysed using an illumination intensity less than 250 mW. Although it is not necessary to understand the mechanism of an invention, it is believed that swollen (hypertonic) liposomes require less photochemical conversion to increase the internal osmotic pressure to the rupture threshold.

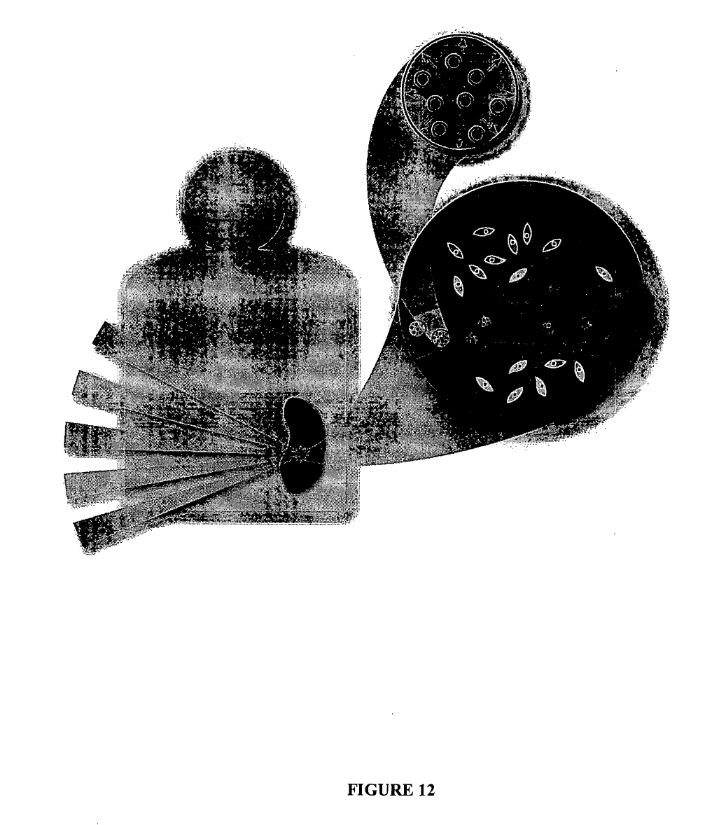

In one embodiment, the present invention contemplates a topologically complex primary liposome population capable of photodynamic lysis induced by radiation ionization effects (i.e., for example, radiation-dynamic effects). It is believed that this capability provides another advantage over conventional photodynamic therapies since radiation ionization emits low power illumination intensities. In one embodiment, the radiation ionization sensitive liposome further comprises a polymer, wherein said polymer degrades upon illumination. In one embodiment, a radiation ionization source comprises an X-ray source. In one embodiment, a radiation ionization source comprises a gamma-ray ray source. Although it is not necessary to understand the mechanism of an invention, it is believed that radiation ionization sources have much greater penetration depth than the 1 centimeter limits of infrared light and have more superior focusing properties. In one embodiment, the topologically complex primary liposome can be administered parenterally and a focused radiation source initiates localized drug release within the illuminated region (i.e., for example, a predefined region; e.g., a lesion identified by previous imaging studies), wherein a therapeutically effective drug is released upon synchronized liposomal lysis. In one embodiment, radiation ionization source illumination can be used to treat deep tissue medical conditions. See, FIG. 12.

Photodynamic therapy utilizing uniform-sized topologically complex primary liposome populations have an important advantage of conventional photodynamic therapies in that they reduce the predominance of drug-induced side effects. Drug side effects are reduced for many reasons including, but not limited to: i) the total amount of photosensitizer it uses is much lower; ii) treated patients do not have to avoid sunlight for 4-6 weeks; and iii) photosensitive uniform-sized topologically complex primary liposomes populations ensures synchronized liposomal lysis, which minimizes the chance that liposomes could escape the illuminated region without releasing the drug. When compared to conventional chemotherapy, the topologically complex primary liposome population delivers the chemotherapeutic drug specifically at the tumor (as opposed to conventional systemic administration of chemotherapeutic drugs), which significantly reduces drug toxicity to normal tissues. Non-lysed liposomes will continue to protect normal tissues from toxic side effects and will be slowly degraded by various clearance mechanisms in the circulation. Overall, delivering therapeutically effective drugs using topologically complex primary liposome populations locally results in a slow general overall distribution in the body and at a lower systemic concentration than in the conventional chemotherapy, thereby resulting in minimal side effects.

### VI. Administration

Some embodiments of the present invention contemplate methods for the treatment of medical diseases (i.e., for example, cancer). In one embodiment, the method provides a photodynamic localized drug release to reduce systemic side effects. For example, broad-spectrum antibiotics, used to treat bacterial infectious diseases, cause many side effects (e.g., diarrhea, caused by the disruption to the intestinal flora), whereas using uniform-sized topologically complex primary liposome photodynamic therapy to delivery broad-spectrum antibiotics reduces these side effects.

Moreover, embodiments of photosensitive topologically complex primary liposome populations can deliver multiple drugs, but also can encapsulate drug-carrying nanoparticles and/or drug-containing secondary liposomes. In one embodiment, the primary liposome encapsulates a secondary liposome, a photosensitizer, and water-soluble drug A, wherein drug B is encapsulated by the secondary liposome. In one embodiment, the illumination of the photosensitive topologically complex primary liposome population results in the lysis of the primary liposome thereby releasing the secondary liposomes and drug A. Although it is not necessary to understand the mechanism of an invention, it is believed that, after primary liposomal lysis, the secondary liposomes containing drug B penetrate a vascular wall of a diseased tissue and slowly release drug B. Consequently, the topologically complex primary liposome population drug delivery system provides a novel and unobvious combination of features including, localized drug release, multi-drug carrying and release characteristics, variable release profiles, thereby offering the potential for a wide range of therapeutic and diagnostic applications.

A. Pulmonary Administration

In one embodiment, the present invention contemplates a method comprising a pulmonary drug delivery system. Although it is not necessary to understand the mechanism of an invention, it is believed that pulmonary administration may be optimized by delivering a uniform-sized liposome population. It is further believed that due to special aerodynamic considerations involved with breathing, particles in a size ranging between approximately 1˜3 μm are most effectively delivered into the deep lung, whereas smaller particles aggregate and may be trapped in the airway. Edwards et al., “Recent advances in pulmonary drug delivery using large, porous inhaled particles” J Appl Physiol 85:379-385 (1998); and U.S. Pat. No. 5,874,064 (herein incorporated by reference). This research suggests that large particles are only useful for facilitating the transportation process into the alveoli. For instance, pulmonary delivery of anti-tuberculosis antibiotics to alveolar macrophages required smaller particles and/or liposomes (<500 nm). Vyas et al., “Aerosolized liposome-based delivery of amphotericin B to alveolar macrophages” Int J Pharm 296:12-25 (2005); and Vyas et al., Design of liposomal aerosols for improved delivery of rifampicin to alveolar macrophages” Int J Pharm 269:37-49 (2004). A further advantage of smaller liposomes is that they have larger surface-to-volume ratio which enhances alveolar wall interactions, thereby increasing tissue uptake rates.

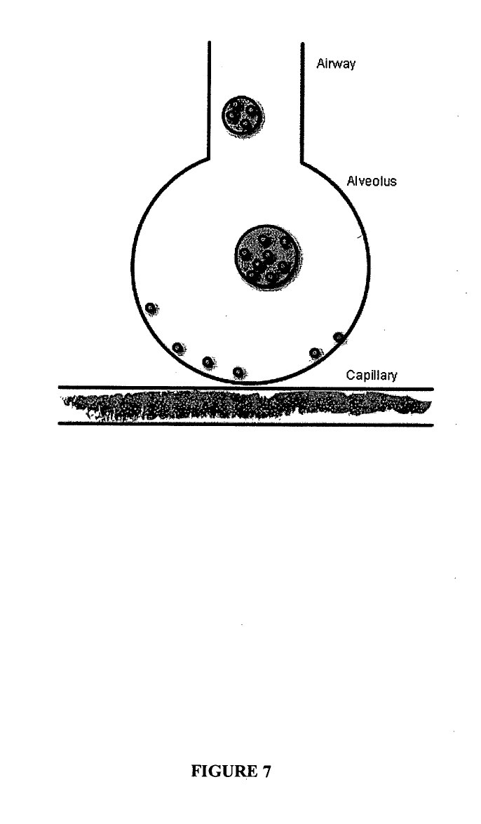

In one embodiment, the present invention contemplates a topologically complex primary liposome comprising a plurality of secondary liposomes of different sizes. Although it is not necessary to understand the mechanism of an invention, it is believed that this size-dependency allows the primary liposome to delivering large liposome complexes and uptake small liposomes, thereby making it a “smart” programmable drug delivery system. See, FIG. 7. In one embodiment, the outer membrane of a topologically complex primary liposome protects a first drug and at least a first secondary liposome population during delivery into the deep lung. In one embodiment, the deep lung surfactants (produced by type II cells) lyse the outer membrane of the primary liposome, thereby releasing the first drug and the secondary liposomes. In one embodiment, the released secondary liposomes are taken up by alveolar macrophages and/or absorbed by the alveolar capillaries. In other embodiments, the surface properties of the larger liposome envelope and internal smaller liposomes could be modified differently, either to enhance or prevent liposome surface adhesion.

### EXPERIMENTAL

### Example I

### Production of a Uniform-Sized Liposome Population

Fatty acid liposomes were prepared by directly dispensing 10 mM oleic acid (NuChek Prep, Inc) in a 0.2 M bicine solution containing 2 mM HPTS (8-hydroxypyrene-1,3,6-trisulfonic acid, a fluorescent dye (Molecular Probes, Eugene, Oreg.) at a final pH of 8.5, vortexed briefly, and tumbled overnight. This procedure resulted in a dye-encapsulated liposome. The methanol is added only to facilitate dye insertion into the liposomal bilayer and is omitted when liposomes are made without fluorescent dye.

POPC liposomes (1-Palmitoyl-2-Oleoyl-sn-Glycero-3-Phosphocholine, Avanti Polar Lipids, Inc.) were prepared in a similar manner by evaporating methanol from a POPC/methanol solution with a rotary evaporator, and rehydrated with 2 mM HPTS 0.2 M bicine solution at a final pH of 8.5 and final concentration of POPC at 10 mM, vortexed briefly, and tumbled overnight. The same buffers without fluorescent dye were used as washing buffers in dialysis. All washing buffers were kept above the critical aggregation concentration, depending on the lipid used.

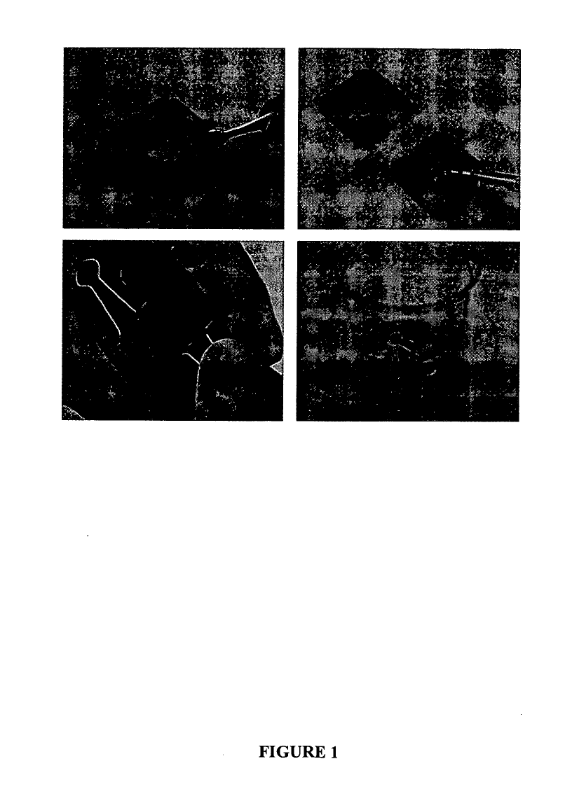

Large-pore dialysis cassettes were modified from commercially available 500 μl dialysis cassettes (Pierce Biotechnology, Inc.). Nuclepore® polycarbonate track-etched membranes (Whatman) were wetted and used to replace the original membranes on the cassette. See, FIG. 1A. Although it is not necessary to understand the mechanism of an invention, it is believed that these polycarbonate track-etched membranes have sharply defined pore sizes and have not been previously used for dialysis.

Liposomes within desired size ranges were made using combinations of differentially sized membrane pores for both the extrusion and dialysis steps. For example, extruding with 5-μm membrane and dialyzing with 3-μm membrane resulted in a liposome diameter range between 3˜5 μm. The extrusion method was similar to those reported previously. Olson et al., “Preparation of Liposomes of Defined Size Distribution by Extrusion through Polycarbonate Membranes” Biochim Biophys Acta 557:9-23 (1979); Hope et al., “Production of Large Unilamellar Vesicles by a Rapid Extrusion Procedure—Characterization of Size Distribution, Trapped Volume and Ability to Maintain a Membrane-Potential” Biochim Biophys Acta 812: 55-65 (1985); and Hanczyc et al., “Experimental models of primitive cellular compartments: Encapsulation, growth, and division” Science 302:618-622 (2003).

A 300˜400 μl extruded dye-encapsulating liposome sample was loaded to the center of the dialysis cassette, after which the cassette was closed by clamps. See, FIGS. 1B & 1C. For each round of dialysis, approximately 30 ml of washing buffer was placed in a 150 ml cup such that the dialysis cassette was just submerged and shaken at approximately 60 rpm. See, FIG. 1D. Shaking was observed to obtain optimal dialysis efficiency, presumably because the fluidic shearing in this setup speeds up the exchange of liposomes through the membrane pores. The first 5-6 rounds of dialysis were performed for approximately 5˜10 min each, after which the free dye in the solution was adequately diluted. Nevertheless, at least 6 more rounds (each at 2 hours minimum) were further performed to sufficiently eliminate liposomes smaller than the membrane pores (One of the 6 rounds was overnight; e.g. approximately 14 hours). The sample was retrieved with a pipette tip by breaking the membrane after dialysis. The sizes of fluorescent-dye-encapsulating liposomes were analyzed by digital fluorescence microscopy (Nikon TE2000S) and Phylum software (Improvision).

### Example H

### Production of a Topologically Complex Liposome Population

Oleic acid (Nu-chek Prep, Inc, Elysian, Minn.) was codissolved with 0.5 mol % rhodamine-DHPE (an anchored membrane dye, Molecular Probes, Eugene, Oreg.) in methanol, which was removed by rotary evaporation followed by resuspension of the thin film by adding 0.2 M bicine buffer at pH 8.5, then vortexed briefly, and tumbled overnight. The methanol is added only to facilitate dye insertion into the liposomal bilayer and is omitted when liposomes are made without rhodamine-DHPE. These rhodamine-DHPE labeled liposomes were extruded with 11 passes by using a stack of two Nuclepore® polycarbonate track-etched membranes with 100 nm diameter pores (Mini-Extruder System®, Avanti Polar Lipids, Inc.) (3-5) to create a set of secondary liposomes.

Subsequently, additional neat oleic acid (i.e., for example, by increasing the final concentration of oleic acid by 10 mM) was added to the secondary liposomes, vortexed and tumbled briefly. This created a topologically complex liposome set comprising primary liposomes encapsulating the secondary liposomes.

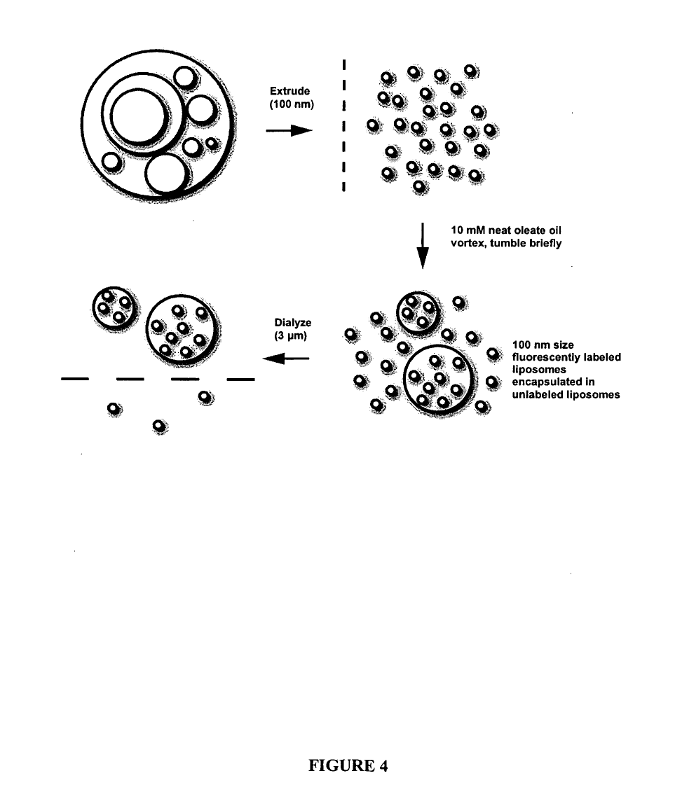

The topologically complex liposome set was then dialyzed with pH 8.5 0.2 M bicine buffer containing 0.8 mM oleic acid using a 3 μm-pore-size cassette (a modified 500 μl dialysis cassette; Pierce Biotechnology, Inc.). Dialysis rounds were performed using sequential 30 ml volumes of washing buffer, which just submerged the laid-down dialysis cassette in a 150 ml beaker, with shaker speed set to 60 rpm. The first 5 rounds of dialysis were performed having a duration of approximately 5˜10 min each, wherein 7 additional rounds of dialysis were performed having a minimum of 2 hours. This protocol sufficiently eliminated liposomes smaller than the membrane pores, including the residual 100 nm diameter secondary rhodamine-DHPE labeled liposomes that were not encapsulated by primary liposome. See, FIG. 4.

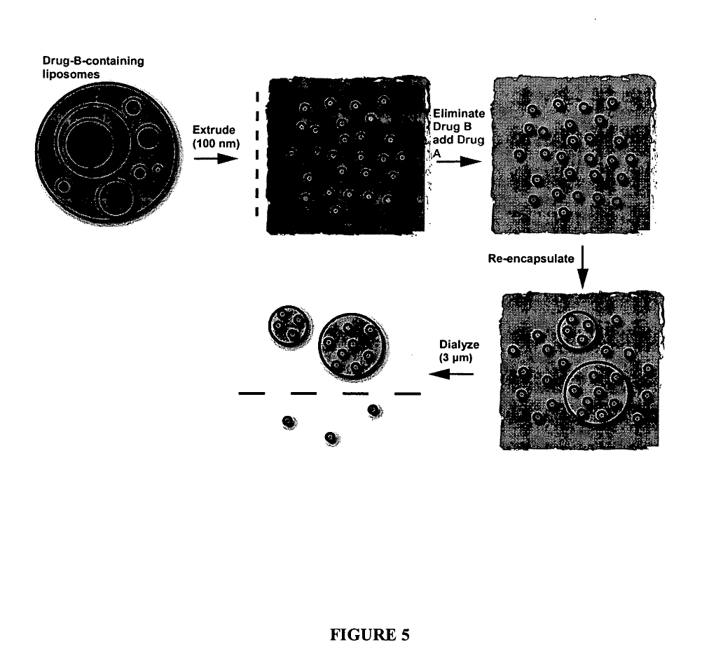

The procedure described above can easily be adapted for making multi-drug delivery liposomes by substituting a drug for the rhodamine dye when preparing the secondary liposomes and adding a second drug to the solution for preparing the primary liposomes. For example, a preparation of multilamellar liposomes containing drug B are extruded to a diameter of 100 nm to create a set of secondary liposomes comprising drug B. Unencapsulated drug B may then be eliminated from the secondary liposome population by size-exclusion chromatography and/or dialysis. Thereafter drug A is added to the additional oleic acid solution such that when the secondary liposomes are re-encapsulated by the formation of the primary liposomes drug A is also encapsulated within the interior of the primary liposome (but not within the interior of the secondary liposomes). Large-pore dialysis is then performed to eliminate both the unencapsulated drug-A-containing secondary liposomes and free drug A still in the solution. See, FIG. 5.

### Example III

### Membrane Disruption of a Primary Liposome

This example shows that an encapsulated internal smaller liposome (i.e., for example, a secondary liposome) can be released from a primary liposome upon controlled lysis of the primary liposomes' outer membrane layer. A slide was prepared having a preparation of a topologically complex liposome population created in accordance to Example II. A drop of 50% NaOH was added on the edge of the slide, which gradually increased the pH in the buffered solution. It was observed that the resultant high pH destroyed the outer membrane of the primary liposome, leading to the release of the small encapsulated liposomes.

### Example IV

### Synchronized Lysis of Photosensitive Topologically Complex Liposomes

Uniform-sized oleic acid liposomes encapsulating 10 mM HPTS were made according to Example II. A slide was prepared by loading 2 μl sample on a 20×60 mm coverslip sealed with an 18×18 mm coverslip and nail polish. An inverted microscope with 60× oil immersion lens (TE2000S, Nikon Inc.) and Phylum software (v3.7, Improvision Inc.) were used for imaging and post-processing. The light source was a 120 W metal halide lamp (EXFO X-cite 120, EXFO Inc.) attached to a 480±20 nm optical filter (Chroma Technology Corp., VT). The light intensity was controlled by a set of two neutral density filters on the microscope.

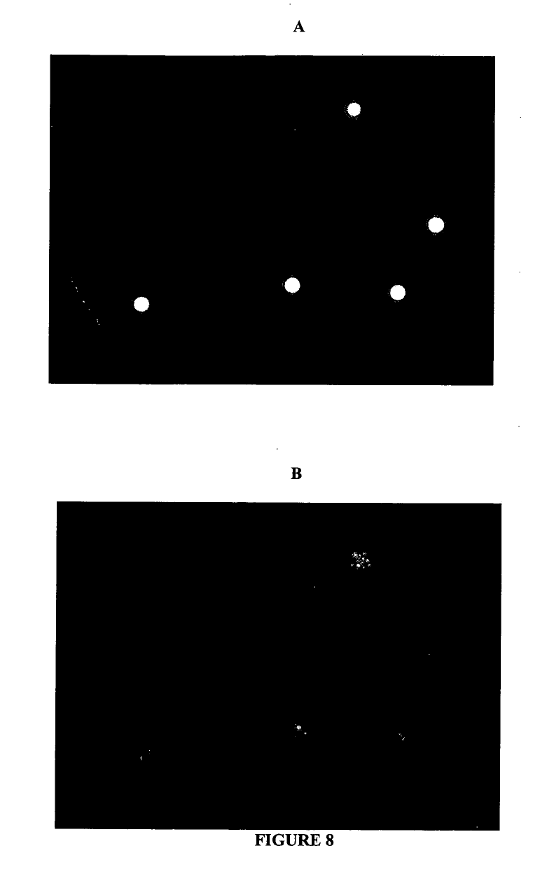

A sample of uniform-sized oleic acid liposomes containing 10 mM HPTS were “exploded” under the photoillumination. The measured time delay between the illumination and explosion events was less than 0.4 sec. Consequently, synchronization of liposomal population lysis denotes high controllability, which is ideal for localizing drug release. See, FIG. 8.

### Example V

### pH Mediation of Synchronized Liposomal Lysis

Dithithreitol (DTT) (Sigma-Aldrich, Inc.), a strong reducing agent, was added to a second primary liposomal sample prepared in accordance with Example IV. When illuminated, the DTT liposome population did not undergo lysis. This observation suggested that reactive oxygen radicals might be involved in the photoreaction.

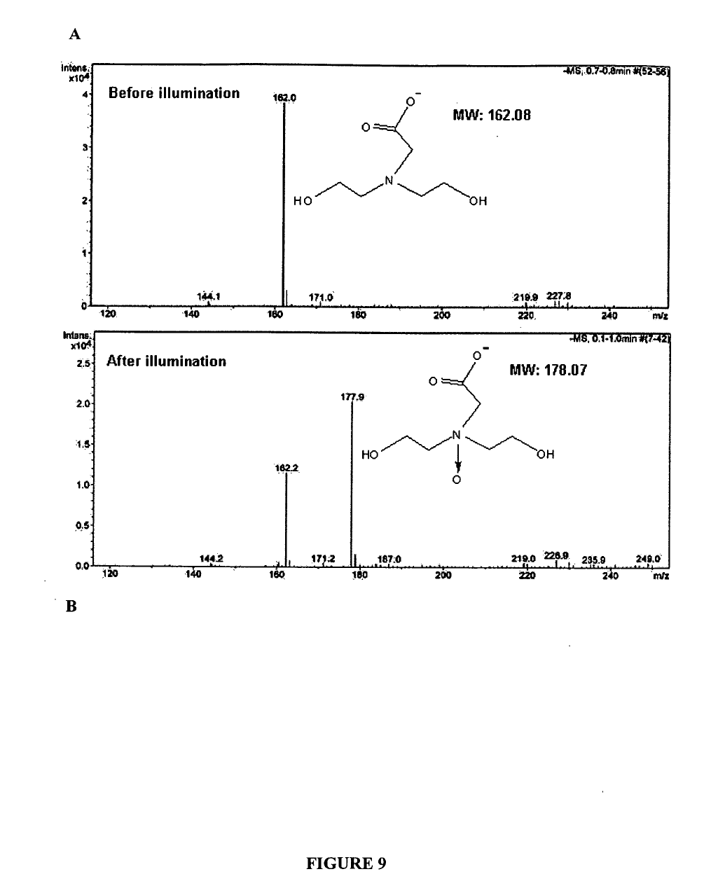

This hypothesis was investigated by placing a 0.2 M pH 8.5 bicine buffer, 2 mM HPTS, and 80 mM H₂O₂ into a 1.5 ml eppendorf tube and illuminating the mixture in accordance with Example IV. The reaction product was analyzed by mass spectroscopy which identified the radical-oxygenation product of bicine. See, FIG. 9.

A 1.5-unit pH drop was also detected in the liposomal solution after illumination. Although it is not necessary to understand the mechanism of an invention, it is believed that the pH drop upon illumination causes an influx of Na⁺ ion into the liposome thereby resulting in an increase of internal osmotic pressure. It is further believed that when the increased osmotic gradient overcomes the membrane surface tension, the liposomes lyse. This hypothesis was confirmed by observing liposomal lysis following injection of a hypotonic solution (data not shown).

### Example VI

### Lipid Bilayer Composition Controls Sequential Temporal Lysis of Liposome Populations

This example predicts that a primary liposome population having a first membrane lipid composition lyses before a secondary liposome population having a second lipid composition.

### Example VII

### Synchronized Lysis of A Topologically Complex Liposome Population Provide Improved Delivery And Efficacy Over Traditional Photodynamic-Induced Release of Liposome Encapsulated Drugs

Photodynamic-induced release of drugs and/or secondary liposomes from a topologically complex liposome population will be compared to provide superior treatment when compared to techniques using modified liposomes. Synchronized lysis will be seen to provide superior treatment to traditional photo-dynamic induced release of drugs from liposomes.

### Example VIII

### An Organic Solvent-Free Topologically Complex Liposome Population

Topologically complex liposome populations produced by the methods described herein are analyzed showing that organic solvents were not detectable in the liposome population and compared to liposome populations prepared by methods which evaporate organic solvents from the liposome preparation. Topologically complex liposome populations will be shown not to contain any organic solvents.

### Example IX

### Topologically Complex Liposomes Provide Improved Drug Delivery and/or Clinical Efficacy

Drug delivery efficiency and/or clinical efficacy will be compared between solvent-containing liposome populations and topologically complex liposome populations (i.e., for example, solvent-free liposomes). Topologically complex liposomes will be shown to provide higher drug delivery efficiency due to their specific localized release characteristics. Topologically complex liposomes will be shown to provide improved clinical efficacy because of the absence of toxic organic solvents.

### Example X

### Synchronized Lysis of Phospholipid Liposomes

This example demonstrates that liposomes having a phospholipid composition, as opposed to fatty acid composition (i.e., for example, oleic acid) undergoes photodynamic synchronized lysis.

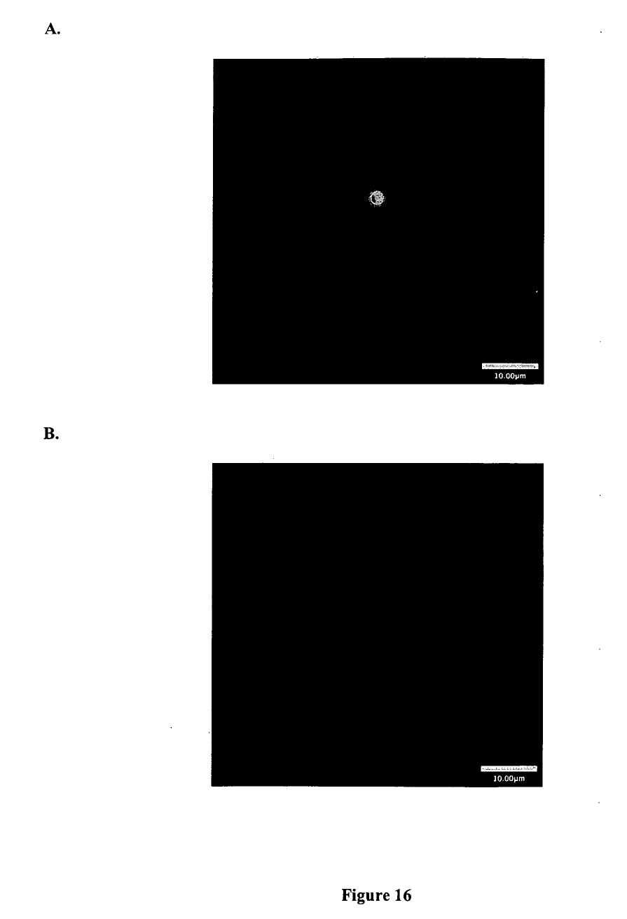

Phospholipid liposomes were prepared by resuspending 10 mM 1-palmitoyl-2-oleoyl-sn-glycero-3-phosphocholine (POPC) in 0.2 M bicine (pH 8.5), containing 10 mM encapsulated HPTS (8-hydroxypyrene-1,3,6-trisulfonic acid trisodium salt). The liposomes were then illuminated under a Nikon TE2000S inverted epifluorescence microscope attached to a metal halide lamp (EXFO, Canada) with a 480±20 nm optical filter (Chroma, Rockingham, Vt.). Shortly after the illumination (˜0.5 sec), the vesicles exploded, releasing the encapsulated internal vesicles See, FIG. 16.

Similar results were obtained using 1,2-Dioleoyl-sn-glycero-3-phosphocoline (DOPC) vesicles (data not shown).

We claim:

1. A topologically complex liposome comprising a primary liposome encapsulating a first drug and a secondary liposome population, wherein said secondary liposome population encapsulates a second drug.

2. The liposome of claim 1, wherein said primary liposome further encapsulates a photosensitizer.

3. The liposome of claim 1, wherein said secondary liposome comprises a bilayer membrane, wherein said first drug is segregated from said second drug by said membrane.

4. The liposome of claim 1, wherein said primary liposome comprises a bilayer membrane, wherein a targeting moiety is attached to said membrane.

5. The liposome of claim 4, wherein said secondary liposome bilayer membrane and said primary liposome bilayer membrane comprise different lipid compositions.

6-10. (canceled)

11. A method, comprising:
a) providing;
i) a multilamellar lipid liposome comprising a first lipid membrane material and a first drug;
ii) a second lipid membrane material; and
iii) a second drug;
b) extruding said multilamellar liposome to create a secondary liposome population comprising said first lipid membrane material and having a maximum average diameter;
c) dialyzing said secondary liposome population, wherein said secondary liposome population further comprises a minimum average diameter; and
d) encapsulating said secondary liposome population with said second lipid membrane material composition and said second drug to form a topologically complex liposome composition comprising a primary liposome population comprising said second lipid membrane material thereby encapsulating said secondary liposome population and said second drug.

12. The method of claim 11, further comprising dialyzing said topologically complex liposome composition, wherein unencapsulated secondary liposomes are removed from said composition.

13. The method of claim 11, wherein said first lipid membrane material and said second lipid membrane material are identical.

14. The method of claim 11, wherein said first lipid membrane material and said second lipid membrane material are different.

15. The method of claim 11, wherein said primary liposome population is of uniform size.

16. The method of claim 11, wherein said secondary liposome population is of uniform size.

17. A method, comprising:
a) providing;
i) a topologically complex liposome composition comprising a primary liposome population encapsulating a secondary liposome population and a drug;
ii) a light source, wherein said light source is capable of inducing a synchronized lysis of said primary liposome population;
b) illuminating said primary liposome population with said light source, thereby inducing a synchronized lysis of said primary liposome population.

18. The method of claim 17, wherein said lysis is mediated by an increase of internal osmotic pressure within said primary liposome population.

19. The method of claim 17, wherein said synchronized lysis of said primary liposome population is complete within 0.4 seconds.

20. The method of claim 17, wherein said synchronized lysis of said primary liposome population releases said drug and said second liposome population.

21. The method of claim 18, wherein said internal osmotic pressure increase is mediated by a pH drop within said primary liposome population.

22. The method of claim 21, wherein said pH drop is mediated by the oxidation of bicine from photooxidation within said primary liposome population.

23. The method of claim 17, wherein said lysis is caused by pH-sensitive primary phospholipid liposomes responding to light-triggered internal pH drop.
